(19) United States
(12) Patent Application Publication
(10) Pub. No.: US 2023/0174965 A1
(43) Pub. Date: Jun. 8, 2023

(54) METHODS AND PRODUCTS

(71) Applicant: THE UNIVERSITY OF BIRMINGHAM, Birmingham (GB)

(72) Inventors: Francisco FERNANDEZ-TRILLO, Edgbaston (GB); Timothy William OVERTON, Edgbaston (GB); Pavan Rao ADONI, Edgbaston (GB); Omar Nicholas Edward HUNEIDI, Edgbaston (GB)

(21) Appl. No.: 17/919,114

(22) PCT Filed: Apr. 16, 2021
(86) PCT No.: PCT/GB2021/050918

§ 371 Date: Oct. 14, 2022

(30) Foreign Application Priority Data
Apr. 17, 2020 (GB) 2005605.7

(51) Int. Cl. C12N 11/08; C12N 1/20

(52) U.S. Cl. CPC C12N 11/08; C12N 1/20; C08F 126/00

(57) ABSTRACT

A method of producing a biofilm-like microorganism-polymer complex, the method comprising the step of exposing, in an aqueous medium, cells of a microorganism to a polymer comprising groups as defined by formula (la) or formula (lb), wherein: Y comprises an imine; and R² comprises a CI-30 group. A biofilm-like microorganism-polymer complex comprising cells of a microorganism and a polymer comprising groups as defined by formula (la) and/or formula (lb). A method for producing a chemical and/or biological product using the microorganism-polymer complex, and the chemical and/or biological products obtained therefrom. A method of dispersing the microorganism-polymer complex. Polymers of formula (la) and of formula (lb).

### FIELD

The present invention relates to the production and/or dispersal of biofilm-like complexes. The present invention provides a method of aiding the formation of a biofilm of a microorganism that employs a polymer that may act as an adhesive scaffold for producing a biofilm-like “microorganism-polymer complex”. The present invention also provides a microorganism-polymer complex. The presently claimed invention defines a method of producing a chemical product, such as a pharmaceutical intermediate, a drug product, or another chemical product using the microorganism-polymer complex. The present invention also defines a method of dispersing the microorganism-polymer complex by cleaving the polymer. The present invention also defines novel polymers.

### BACKGROUND

Some microorganisms readily form biofilms. As compared to being in their planktonic form, microorganisms in biofilms may be more resilient to certain environmental conditions, such as extremes of pH or high temperatures. For example, a biofilm of a microorganism may be more resilient to chemical attack than a planktonic form of the microorganism. This may be due to the microenvironment within the biofilm shielding certain parts of the biofilm from chemical attack.

Chemical products, such as fine chemicals and/or pharmaceuticals, may be synthesised using biologically-mediated synthesis. For example, microorganisms can be used to perform chemical transformations or biosyntheses to produce products of commercial value. Biofilms have been used to mediate the synthesis of chemical products, as described in Biofilm Bioprocesses; F. Costa et al.; Current Developments in Biotechnology and Bioengineering; 2017; 143-175; doi: 10.1016/B978-0-444-63663-8.00006-9.

In terms of benefits to a biologically-mediated process, the use of a microorganism in a biofilm rather than the same microorganism in a planktonic form may increase the resilience of the microorganism, may produce a higher yield of a desired product, and/or may produce fewer and/or a lower yield of by-products.

However, not all microorganisms may naturally form biofilms. Some microorganisms may form biofilms under some conditions but not under others. Some microorganisms may form small and/or weak, e.g. less mechanically, chemically and/or thermally stable, biofilms.

Therefore, it would be beneficial to enable microorganisms that do not naturally form biofilms to obtain one or more benefits similar to those attained by a biofilm. It would be beneficial to enable microorganisms that only form biofilms under certain conditions to attain one or more benefits similar to those attained by a biofilm under a broader range of conditions. It would be beneficial to enable microorganisms to form larger and/or stronger, e.g. more mechanically, chemically and/or thermally stable, biofilm-like structures.

The present invention has been devised with the foregoing in mind.

### SUMMARY

According to a first aspect, the present invention provides a method of producing a microorganism-polymer complex, the method comprising the step of exposing, in an aqueous medium, cells of a microorganism to a polymer comprising groups as defined by formula (Ia) or formula (Ib):

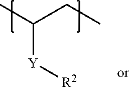

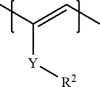

wherein:
Y comprises an imine; and
R² comprises a C1-30 group.

Surprisingly, it has been determined that polymers as defined by formula (I), including those of formulae (Ia) and (Ib), which contain a C1-30 group, may facilitate the production of the microorganism-polymer complex. Surprisingly, it has been found that the microorganism-polymer complex may exhibit similar properties to those of a biofilm.

The method of the first aspect may enable a microorganism that does not form a biofilm to access benefits obtainable from being able to form a biofilm.

The method of the first aspect may enable a microorganism that does not form a biofilm under certain conditions to access benefits from being able to form a biofilm under those certain conditions.

The method of the first aspect may enable a microorganism to form a larger and/or stronger, e.g. more mechanically, chemically and/or thermally stable, biofilm-like complex in the form of a microorganism-polymer complex.

The method of the first aspect may facilitate the formation of a biofilm-like environment, via the microorganism-polymer complex, for the cells of the microorganism to enable the user to attain benefits obtainable from using a biofilm. The formation of the biofilm-like environment may allow a process using the microorganism to be performed under different conditions, such as more harsh conditions, that may enhance characteristics of the process, such as increase the yield of a desired product, or produce fewer and/or a lower yield of by-products.

Without being limited by theory, it is thought that the polymer, perhaps specifically the R² group, may adhere to regions of the cell walls of the microorganisms, such as hydrophobic regions. The polymer backbone (R¹) and linker groups (Y) hold the C1-30 groups (R²) together, so the polymer may, therefore, define a scaffold upon which cells of the microorganism can attach. Thus a complex of the microorganism and the polymer (i.e. the microorganism-polymer complex) may be formed. This may provide a local environment that is similar to that within a biofilm for that microorganism. In one embodiment the R² group is hydrophobic, for example having a clogP and/or clogD of -1.0 or higher.

Where cells of the microorganism have attached to the polymer they may have a reduced freedom of movement, allowing for interactions to form between the cells, and between the attached cells and unattached cells, to form a microorganism-polymer complex. Thus, the polymer may act as a “seed” upon which a microorganism can attach, to form a microorganism-polymer complex; and the microorganism-polymer complex may act as a biofilm, and/or as a “seed” upon which a biofilm can form. Thus, the microorganism may express biofilm-like characteristics.

It will be appreciated that the surface characteristics of cells, for example charge and/or hydrophobicity, can differ between species of microorganisms, and may also differ between strains of the same species of microorganism. As such, whilst one R² group may be optimal for production of a microorganism-polymer complex for one microorganism, a different R² group may be optimal for another organism.

A benefit of compounds of formulae (Ia) or (Ib) including an imine - e.g. a hydrazone, such as an acyl hydrazone, or an oxime, or another group as shown in FIG. 1, - is that this enables rapid screening of a variety of R² groups. It has been determined that the synthesis of polymers with imine groups is relatively facile. Therefore, synthesis of polymers containing a group as defined by formula (I), e.g. (Ia) or (Ib), and wherein the Y group contains an imine, e.g. an acyl hydrazone or another group as shown in FIG. 1, may allow for easy and/or rapid screening of a variety of R² groups. This may be used to facilitate a search to find an optimal R² group for any particular microorganism.

The present invention provides, according to a second aspect, a microorganism-polymer complex. The complex comprises cells of a microorganism and a polymer comprising groups as defined by formula (Ia) and/or formula (Ib). In one embodiment the microorganism-polymer complex is produced by (obtainable by), e.g. obtained by, the method of the first aspect.

It has surprisingly been found that the microorganism-polymer complex of the present invention can have particular application in synthesis. For example, the microorganism-polymer complex can be used in the synthesis of a medicament, such as an organic molecule, or protein binding fragment, such as an antibody fragment. The microorganism-polymer complex may be used in the synthesis of a bulk and/or fine chemical.

The present invention provides, according to a third aspect, a method for producing a chemical and/or biological product, the method comprising exposing a substrate to a microorganism-polymer complex as defined by the second aspect. It will be understood that this step should be performed so as to allow the complex to react with the substrate. According to a fourth aspect, the present invention provides a chemical and/or biological product obtainable by (produced by) the third aspect.

In one embodiment the chemical and/or biological product is a medicament, such as an active pharmaceutical ingredient, or an intermediate thereof. The chemical and/or biological product may be an organic molecule, a protein binding fragment and/or an antibody. The chemical and/or biological product may be a fine chemical and/or a bulk chemical.

It has surprisingly been found that the microorganism-polymer complex of the invention can be controllably dispersed e.g. where cells of the complex are released from one another and/or the polymer. For example, this may be useful for controlling the rate at which a reaction mediated by the complex proceeds, for controlling the growth of the cells of the complex, for controlling the growth of the complex itself, and/or for controlling the amount of product produced. Disrupting the microorganism-polymer complex may be useful when terminating a reaction performed by the microorganisms in the microorganism-polymer complex. This may be useful when cleaning apparatus which has been used to perform such a reaction.

According to a fifth aspect, the present invention provides a method of dispersing a microorganism-polymer complex, wherein the microorganism-polymer complex is of the second aspect, wherein the polymer of the microorganism-polymer complex is cleavable, and wherein the method comprises the step of cleaving the polymer.

The polymer may be cleavable. It will be understood that the polymer effectively holds cells together in the complex, and so cleaving the polymer will reduce the cohesion between cells. This may allow the cells to fall away from one another, naturally and/or with agitation e.g. stirring, allowing the microorganism to change into a planktonic form. This may provide the benefit that the microorganism-polymer complex can be dispersed. The R² group may be cleavable from the polymer backbone. Preferably Y is cleavable. Y may be cleavable into a first region, which is attached to the polymer backbone, and a second region, which is attached to the R² group. Preferably cleavage is performed by hydrolysis.

The imine functional group, e.g. acyl hydrazone or each other group as shown in FIG. 1, also has the benefit that it may be cleavable. For example, it may be cleavable under conditions that can be described as “orthogonal” in that they are not deleterious to bacterial life. Cleavage may be performed by modifying the pH and/or adding a competing agent, such as a primary acylhydrazine, for example at an acidic pH, e.g. from 2 to 7, for example from 4 to 6.5 (Park, K.D. et al., Chemistry & Biology, Volume 16, Issue 7, Pages 763-772).

It has surprisingly been determined that the polymer may be used to controllably form a microorganism-polymer complex. For example, the polymer may be used to form a microorganism-polymer complex, and then the complex may be dispersed, and then the complex may be reformed.

The present disclosure relates to methods employing polymers containing a group of formula (I), e.g. (Ia) or (Ib). According to a sixth aspect, the present invention provides a polymer containing a group as defined by formula (Ia) or formula (Ib), wherein R² is selected from the list consisting of:

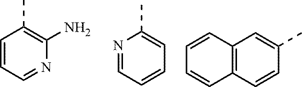

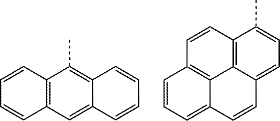

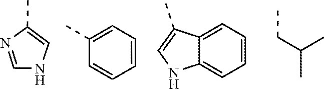

with the proviso that if the group is of formula (Ia′):

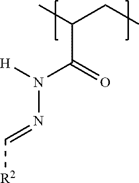

then R² must be selected from the list consisting of:

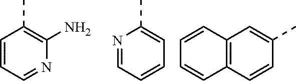

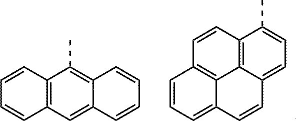

In one embodiment R² is selected from the list consisting of:

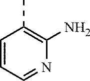

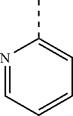

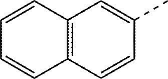

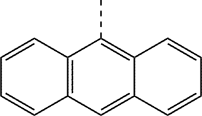

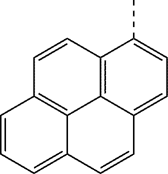

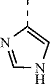

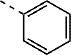

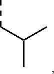

with the proviso noted above.

As discussed in the Examples section, it has been determined that polymers containing units with such R² groups have particularly beneficial properties in terms of assisting biofilm formation.

The polymer of the sixth aspect may contain a group as defined by the following formula selected from the list consisting of:

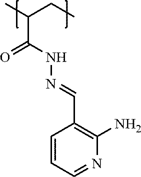

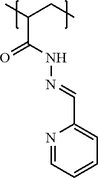

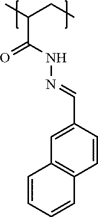

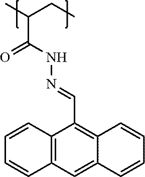

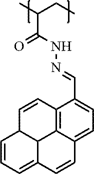

The complexes of the present invention may find particular use as coatings for articles such as, in agricultural applications, seeds. Thus, according to a seventh aspect, the present invention provides a coated article comprising an article coated with a microorganism-polymer complex according to the second aspect. The article may be a seed, such as a seed of a plant in the family Fabaceae.

The complexes of the present invention may find particular use as treatments for soil. Thus, according to an eighth aspect, the present invention provides soil comprising a microorganism-polymer complex according to the second aspect.

The complexes of the present invention may also find particular use in sustaining or promoting the growth of microorganisms beneficial for the skin, buccal cavity (mouth) or gut, as either an oral formulation or as a topical formulation. Thus, according to a ninth aspect, the claimed invention provides a composition for oral administration or topical application, wherein the composition comprises a microorganism-polymer complex of the second aspect. The composition may be a pharmaceutical composition and/or a cosmetic formulation. The composition may include a carrier, for example a dermatologically safe, edible and/or pharmaceutically acceptable carrier.

The subject matter of the following clauses is also provided: 1. A method of producing a microorganism-polymer complex, the method comprising the step of exposing, in an aqueous medium, cells of a microorganism to a polymer comprising groups as defined by formula (I):

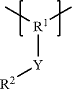

wherein:R¹ comprises a unit of a polymer backbone; Y comprises a linker group; and R² comprises a C1-30 group.

2. A microorganism-polymer complex comprising cells of a microorganism and a polymer comprising groups as defined by formula (I).

3. The microorganism-polymer complex of clause 2, wherein the complex is obtainable by the method of clause 1.

4. The method of clause 1 or the complex of clause 2 or clause 3, wherein the polymer is cleavable.

5. The method of clause 1 or clause 4, or the complex of any one of clauses 2 to 4, wherein Y comprises one or more groups selected from the list of: amide (such as a secondary or tertiary amide), ester, amine (such as a secondary or tertiary amine), ether, dialkyl peroxide, thioether, disulfide, sulfoxide, sulfone, sulfonamide, sulfonate ester, thioketone, thioester, phosphine, phosphonate ester, phosphate ester, boronic ester, borinic ester, borane, ketone, carbamate, carbonate, carboxylic acid anhydride, urea, ketal, acetal, orthoester, orthocarbonate, imine (e.g. an oxime and/or a Schiff base such as a secondary ketamine and/or a secondary aldimine), imide, diimide, hydrazine and 1,2,3-triazole.

6. The method or the complex of clause 5, wherein Y comprises one or more groups selected from the list of: amide, ester, carboxylic acid anhydride, carbamate, carbonate, urea, imine, hydrazine and 1,2,3-triazole.

7. The method or the complex of clause 6, wherein Y comprises a hydrazone.

8. The method of any one of clauses 1 and 4 to 7, or the complex of any one of clauses 2 to 7, wherein R² comprises a C3-30 group.

9. The method or the complex of clause 8, wherein R² comprises a C5-30 group.

10. The method or the complex of clause 9, wherein R² comprises a C5-30 aryl or heteroaryl group.

11. The method of any one of clauses 1 and 4 to 10, or the complex of any one of clauses 2 to 10, wherein the clogD and/or clogP of the R² group is -2.0 or higher.

12. The method of any one of clauses 1 and 4 to 11, or the complex of any one of clauses 2 to 12, wherein the microorganism is a bacteria or a fungi.

13. The method or the complex of clause 12, wherein the microorganism is a bacteria.

14. A method for producing a chemical and/or biological product, the method comprising exposing a substrate to a microorganism-polymer complex as defined by any one of clauses 2 to 13.

15. A chemical and/or biological product obtainable by clause 14.

16. The method of clause 14 or the product of clause 15, wherein the chemical and/or biological product is a medicament, an intermediate of a medicament, an organic molecule, a protein binding fragment, an antibody, a fine chemical and/or a bulk chemical.

17. A method of dispersing a microorganism-polymer complex, wherein the microorganism-polymer complex is as defined by clause 4 or any one of clauses 5 to 13 as dependent upon clause 4, and wherein the method comprises the step of cleaving the polymer.

18. The method of clause 17, wherein the polymer is cleaved by hydrolysis.

19. A polymer containing a group as defined by formula (I).

20. The polymer of clause 19, wherein R² is selected from the following group:

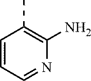

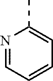

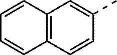

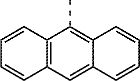

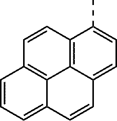

### DETAILED DESCRIPTION

### Polymer

The present disclosure relates to a polymer comprising groups as defined by formula (I):

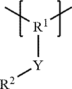

wherein: R¹ comprises a unit of a polymer backbone; Y comprises a linker group; and R² comprises a C1-30 group.

In particular, the claimed invention relates to a polymer comprising groups as defined by formula (Ia) or formula (Ib):

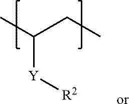

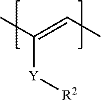

wherein: Y comprises an imine; and R² comprises a C1-30 group. It will be understood that formulae (Ia) and (Ib) are examples of formula (I) and that, unless they are contradictory, descriptions made in relation to formula (I) can apply equally in relation to formulae (Ia) and/or (Ib).

The prefix “CX-Y” (where X and Y are integers) as used herein refers to the range of the number of carbon atoms in a given group. For example, a C1-6 group contains from 1 to 6 carbon atoms, and a C3-6 group contains from 3 to 6 carbon atoms.

The polymer may contain two or more groups defined by formula (I), such as three or more, or 5 or more, or 10 or more, or 20 or more, or 50 or more groups defined by formula (I).

The polymer may contain a percentage of groups defined by formula (I), by weight or moles, of 25% or more, such as 35% or more, or 45% or more. Preferably the percentage of groups defined by formula (I), by weight or moles, is 50% or more, such as 60% or more, or 70% or more. The percentage of groups defined by formula (I), by weight or moles, may be 80% or more, or 90% or more, such as 95% or more or 98% or more. The polymer may contain a percentage of groups defined by formula (I), by weight or moles, of from 25% to 99%, such as from 35% to 98%, or from 45% to 97%. For example, the percentage of groups defined by formula (I), by weight or moles, may be from 50% to 95%, such as from 50% to 90%, or from 50% to 80%. In one embodiment the polymer consists essentially of, or consists of, groups as defined by formula (I).

The molar percentage of the groups defined by formula (I) may be determined by comparison of the integration of ¹H NMR peaks.

It will be understood that the polymer may also contain groups that do not fall within the definition of formula (I). For example, the polymer may contain one or more groups as defined by formula (II):

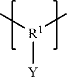

for example one or more groups as defined by formula (IIa) or (IIb):

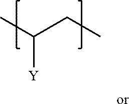

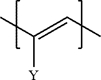

Wherein, as with or independently of formula (I): R¹ is a unit of a polymer backbone; and Y is a linker group.

For formula (II), Y may comprise an imine, or an amine, hydroxylamine, hydrazine, acyl hydrazine, acylthiohydrazine, semicarbazine, semithiocarbazine, hydrazine carboximidamide, carbazate hydrazine, thiocarbazate hydrazine, dithiocarbazate hydrazine, carbazide, thiocarbazide, azacarbazide, 2-hydrazideyl pyridine, 2-hydrazideyl pyrimidine and 2-hydrazideyl triazine, ketone or aldehyde.

It will be understood that the Y of the group of formula (II) may include one two or more atoms and/or groups, e.g. hydrogen atoms, that are not present in the Y of formula (I) to take the place of the R² group. The polymer may be a co-polymer, such as a random or block co-polymer. Usually the R¹ group of formula (I) will be the same as the R¹ group of formula (II)

The polymer may contain a percentage of groups defined by formula (II), by weight or moles, of 75% or less, such as 65% or less or 55% or less. Preferably the polymer contains a percentage of groups defined by formula (II), by weight or moles, of 50% or less, such as 40% or less, or 30% or less. In one embodiment the polymer contains a percentage of groups defined by formula (II), by weight or moles, of 20% or less, such as 10% or less, or 5% or less. The percentage may be from 1% to 75%, such as from 2% to 50% or from 5% to 40%.

The polymer may consist essentially of, or consist of groups defined by formula (I) and formula (II). The polymer may contain a percentage of groups defined by formula (I) or formula (II), by weight or moles, of 50% or more, or 60% or more, such as 70% or more, or 80% or more. Preferably the polymer contains a percentage of groups defined by formula (I) or formula (II), by weight or moles, of 85% or more, or 90% or more, such as 95% or more, or 98% or more. The percentage may be from 50% to 99%.

In one embodiment the polymer contains a percentage of groups defined by formula (I) or formula (II), by weight or moles, of 60% or more, and contains a percentage of groups defined by formula (I), by weight or moles, of 25% or more. For example, the polymer may contain a percentage of groups defined by formula (I) or formula (II), by weight or moles, of 80% or more, and contain a percentage of groups defined by formula (I), by weight or moles, of 50% or more.

The polymer may be solid (e.g. a powder, plates, needles, beads, and/or a block) or liquid. Preferably the polymer is a solid (e.g. powder, plates, needles and/or beads) that is soluble and/or suspendable in the aqueous medium.

The number average molecular weight (Mₙ) of the polymer may be 1 kDa or more, such as 2 kDa or more, or 3 kDa or more, or 4 kDa or more. Preferably Mₙ is 5 kDa or more, such as 6 kDa or more, or 7 kDa or more, for example 8 kDa or more. In one embodiment Mₙ may be 10 kDa or more, or 20 kDa or more, or 50 kDa or more, such as 100 kDa or more, or 500 kDa or more. In one embodiment Mₙ of the polymer is 5 MDa or less, such as 2 MDa or less, or 1 MDa or less. For example, Mₙ may be 500 kDa or less, such as 250 kDa or less, or 100 kDa or less, for example 75 kDa or less, or 60 kDa or less, such as 50 kDa or less. In one embodiment Mₙ of the polymer is from 1 kDa to 5 MDa, such as from 2 kDa to 1 MDa, or from 2 kDa to 250 kDa. For example, Mₙ may be from 3 kDa to 100 kDa or from 4 kDa to 60 kDa. Mₙ may be determined by GPC-SEC in THF using PMMA standards.

Preferably the polymer is not cross-linked.

It will be understood that square brackets used in the chemical formulae of the present disclosure signify the boundaries of a group defined by a formula, wherein the group forms part of a polymer. It signifies that the polymer backbone extends. The polymer may be terminated with any suitable group. The groups at the terminal ends of the polymer chain may depend upon the agent used to polymerise the monomer units. For example, where the monomer is polymerised with 2-(ethylthiocarbonothioylthio)-2-methylpropanoic acid, the polymer chain may be terminated by a 2-(2-methylpropanoic acid) group and either a thiol or an ethylthiocarbonothioylthio group.

### R¹

As defined by formula (I), R¹ is a unit of a polymer backbone. Formula (Ia) defines polyethylene backbones, and Formula (Ib) defines polyacetylene backbones. A key feature of the polymer backbone is that it holds together the Y groups that are attached to the R² groups so that the R² groups can attach to the cells of the microorganism and the polymer holds the cells together to form a microorganism-polymer complex. It will be understood that the structure and/or composition of the polymer backbone may vary, for example due to ease of synthesis.

The person skilled in the art will be able to use their common general knowledge to adapt the present methods to a variety of polymer backbones. For the more broad disclosure of Formula (I), the polymer backbone may be based on any poly(olefin), such as a poly(acrylic) acid, a poly(methacrylic) acid, a poly(acetylene) and/or a poly(styrene), and/or a polymer made by ring opening, such as a poly(ether), a poly(ester), a poly(amino acid), and/or a poly(oxazoline), and/or a polymer made by metal-mediated polymerization, such as a poly(olefin), a poly(acetylene), and/or a poly(isocyanide), and/or copolymers thereof.

R¹ of formula (I) may be a unit of a poly(amino acid) (i.e. polypeptide) backbone. Such backbones have been described in Song, Z. et al., Chem. Soc. Rev., 2017, 46, 6570-6599 and references therein. For example, R¹ may include aspartic acid or glutamic acid unit. Aspartic acid and glutamic acid have pendant carboxylic acid units that may form the linker group Y.

For example, formula (I) may be defined as:

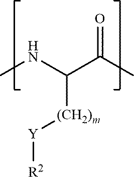

Wherein m is an integer from 0 to 3. In one embodiment, m is 1 or 2. These integers correspond to a backbone formed of aspartic acid or glutamic acid respectively. It will be noted that this does not fall within the scope of formulae (Ia) and (Ib).

R¹ may be a unit of a polyethylene backbone. For example, formula (I) may be defined as:

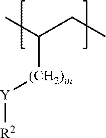

Wherein m is an integer from 0 to 3. In one embodiment, m is 0 or 1. Preferably m is 0, such that formula (I) is defined as:

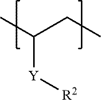

In one embodiment the polymer backbone, including groups of formula (I) and any groups not defined by formula (I), may contain 10 or more carbon atoms, such as 20 or more, or 30 or more. Preferably the polymer backbone contains 40 or more carbon atoms, or 60 or more, such as 80 or more, or 100 or more carbon atoms. The polymer backbone may contain 250 or more carbon atoms, such as 500 or more carbon atoms, for example 1000 or more, or 2500 or more, such as 5000 or more carbon atoms. In one embodiment the polymer backbone includes 10000 carbon atoms or fewer, such as 5000 or fewer, or 1000 or fewer, for example 500 or fewer or 250 or fewer. The polymer backbone, including groups of formula (I) and any groups not defined by formula (I), may contain from 10 to 10000 carbon atoms, such as from 40 to 1000 carbon atoms, for example from 60 to 250 carbon atoms.

### Y

As defined by formula (I), Y comprises a linker group. Formulae (Ia) and (Ib) require that Y comprises an imine.

Imines are functional groups that contain a C═N group, i.e. a carbon atom double bonded to a nitrogen atom. For the compounds of the present invention, the nitrogen should not simply be substituted with a hydrogen; this is made clear by formula (I) having two groups attached to the Y group that contains the imine, namely the polymer backbone and the R² group. The nitrogen of the imine may be substituted with a hydrocarbon group or heteroatoms such as oxygen or nitrogen that are substituted with a hydrocarbon group.

It will be understood that the imine should be positioned such that (hypothetical) cleavage of the imine would detach the R² group from the polymer backbone. As such, it will be understood that the imine of the Y group is positioned between the polymer backbone and the R² group. The imine of the Y/linker group links the polymer backbone to the R² group.

Preferably the nitrogen of the imine is substituted with oxygen or nitrogen that are substituted with a hydrocarbon group. The carbon atom of the imine may be substituted by hydrogen atom(s), hydrocarbon group(s) (e.g. alkyl, aryl, alkenyl, alkynyl, or heterocyclyl) and/or heteroatom (s).

Preferably the imine is part of a wider functional group. The imine may be part of a Schiff base, which is a group that contains a hydrocarbon group directly bonded to the nitrogen atom of the imine. The imine may be part of a group selected from, but not limited to, the list consisting of hydrazone, oxime, acylhydrazone, acylthiohydrazone, semicarbazone, semithiocarbazone, hydrazone carboximidamide, carbazate hydrazone, thiocarbazate hydrazone, dithiocarbazate hydrazone, carbazone, thiocarbazone, azacarbazone, 2-hydrazoneyl pyridine, 2-hydrazoneyl pyrimidine and 2-hydrazoneyl triazine. FIG. 1 of the accompanying drawings depicts the structures of the groups in this list.

Preferably the nitrogen atom of the imine is more closely (but directly or indirectly) bonded to the polymer backbone than the carbon atom, and the carbon atom of the imine is more closely (but directly or indirectly) bonded to the R² group than the nitrogen atom.

In one embodiment Y comprises one or more further groups selected from the list consisting of: amide (such as a secondary or tertiary amide), ester, amine (such as a secondary or tertiary amine), ether, dialkyl peroxide, thioether, disulfide, sulfoxide, sulfone, sulfonamide, sulfonate ester, thioketone, thioester, phosphine, phosphonate ester, phosphate ester, boronic ester, borinic ester, borane, ketone, carbamate, carbonate, carboxylic acid anhydride, urea, ketal, acetal, orthoester, orthocarbonate, imide, diimide, hydrazine, hydroxylamine, 1,2,3-triazole, alkyl, alkenyl, alkynyl, aryl and heterocyclyl.

Y may comprise one or more further groups selected from the list consisting of: amide, ester, ether, carboxylic acid anhydride, carbamate, carbonate, urea, imine, hydrazine, hydroxylamine and 1,2,3-triazole. Preferably Y comprises one or more further groups selected from the list consisting of amine, ether, aryl, heteroaryl and alkyl.

It will be understood that the imine (e.g. one of the imine-containing functional groups disclosed by FIG. 1) of Y may be, but preferably is not, directly bonded to the polymer backbone (R¹). The imine (e.g. one of the imine-containing functional groups disclosed by FIG. 1) of Y may be directly bonded to the R² group. In particular, the Y group may include one or more groups selected from the list in the paragraph above between the imine and the polymer backbone and/or the R² group.

Y of Formula (I) may comprise an amide and/or an imine (e.g. a hydrazone, oxime and/or a functional group disclosed by FIG. 1) moiety. Such groups may allow for rapid screening of a variety of R² groups. Formulae (Ia) and (Ib) require that Y comprises an imine, this may for example be a hydrazone, an oxime and/or a functional group disclosed by FIG. 1.

Y may contain alkyl, alkenyl, alkynyl, aryl and/or heterocyclyl groups. Suitable definitions of these groups are made in relation to R², and these may apply equally to Y. Preferred groups of this nature for Y include C5-10 aryl or heteroaryl groups, such as C6 aryl groups (e.g. phenyl), and/or C1-C6 alkyl groups, such as C1 and/or C2 alkyl groups. Such groups may be linked by heteroatoms such as O, N or S, preferably O.

For example, Y may include one or more, such as from one to ten, for example two, three or four, ether (e.g. ethylene glycol (i.e. —(OCH₂CH₂)ₙO—) or propylene glycol (i.e.—(OCH₂CH₂CH₂)ₙO—)) groups. Preferably Y includes one or more ether (e.g. ethylene glycol or propylene glycol) groups and a phenyl group. For example, Y may include, or be:

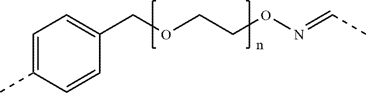

where n is an integer from 0 to 5, for example from 0 to 3.

Preferably Y contains a carbamate or urea group, for example a semicarbazone or a carbazate hydrazone. For example, Y may include, or be:

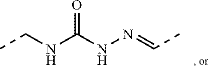

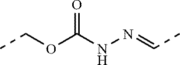

Preferably formula (I) is:

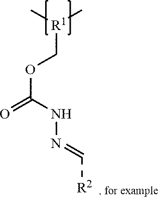

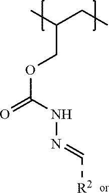

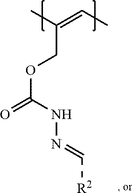

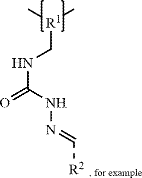

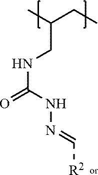

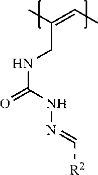

The skilled person will appreciate that a polymer where Y comprises an amide and/or an imine may be synthesised by reacting a compound including a nucleophilic amine, such as a primary amine, a hydrazine and/or an acyl hydrazine with a carbonyl-containing compound. The carbonyl-containing compound may contain an aldehyde, a ketone, a carboxylic acid chloride, a carboxylic acid anhydride, a carboxylic acid ester. The carbonyl-containing compound is preferably a compound containing an aldehyde or a ketone. More preferably the carbonyl-containing compound contains an aldehyde as these compounds more readily form imines, e.g. hydrazones.

Preferably the carbonyl-containing compound includes the R² group.

Preferably Y comprises an acyl hydrazine functional group. It will be understood that an acyl hydrazine is an amide of a hydrazine:

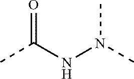

Preferably formula (I) is:

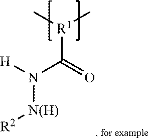

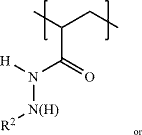

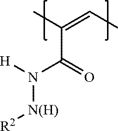

Dashed bonds between two groups (for example between the acyl hydrazine and R²) indicate that R² may be directly or indirectly (e.g. bonded with an intervening methylene group) bonded to the acyl hydrazine. Preferably the dashed bonds indicate direct bonds. For example, there may be a single or a double bond between the N(H) group and R². It will be understood that where a double bond is present between the N and the R² group, the H of the N(H) group is not present.

In one particularly presently preferred embodiment the polymer of formula (I) includes an acyl hydrazone or an acyl hydrazine, e.g:

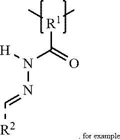

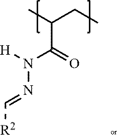

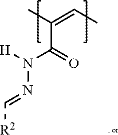

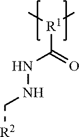

It will be appreciated that such acyl hydrazines may be accessed by reduction of the acyl hydrazone, for example by hydrogenation in the presence of a transition-metal containing catalyst and/or by a reducing agent such as sodium cyanoborohydride. Alternatively, such acyl hydrazines may be accessed by the reaction of a hydrazine with a carboxylic acid derivative, such as a carboxylic acid chloride, a carboxylic acid anhydride, a carboxylic acid ester or similar, to form an amide functional group.

Y may comprise an amine, hydrazine, hydroxylamine (alkoxyamine) and/or a similar moiety. For example, R¹, R² and Y may be bonded in the following manner:

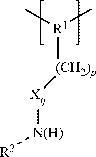

Wherein X is NH, O, PH or S, and wherein q is 0 or 1, and p is an integer from 0 to 20. In one embodiment X is NH or O. In one embodiment, p is an integer from 1 to 10 or from 1 to 6, such as 1 or 2 or 3. In one embodiment X is NH or O and p is an integer from 1 to 3. The dashed bond between the N(H) group and R² indicates that R² may be directly or indirectly bonded to the N(H) group. For example, there may be a single or a double bond between the N(H) group and R². It will be understood that where a double bond is present the H of the N(H) group is not present.

It will be appreciated that such compounds may be accessed by reduction of an amide or imine, for example by hydrogenation in the presence of a transition-metal containing catalyst or by a reducing agent such as sodium cyanoborohydride or lithium aluminium hydride.

In one embodiment Y includes a hydrazine. For example, the group of formula (I) may be:

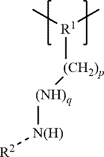

Wherein p is an integer from 0 to 20, and wherein q is 0 or 1. In one embodiment, p is an integer from 1 to 10 or from 1 to 6, such as 1 or 2 or 3.

In one embodiment the group of formula (I) is:

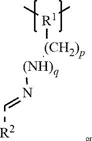

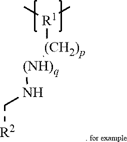

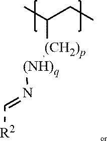

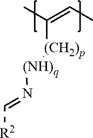

It will be appreciated that hydrazines or amines may be accessed by reduction of a hydrazone or an imine, for example by hydrogenation in the presence of a transition-metal containing catalyst or by a reducing agent such as sodium cyanoborohydride.

In one embodiment Y includes an oxime or hydroxylamine. For example, formula (I) may be:

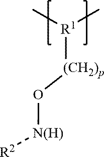

Wherein p is an integer from 0 to 20, and wherein q is 0 or 1. In one embodiment, p is an integer from 1 to 10 or from 1 to 6, such as 1 or 2 or 3.

In one embodiment the group of formula (I) is:

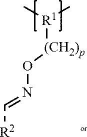

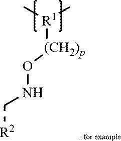

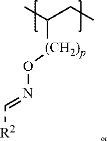

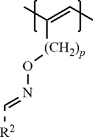

It will be appreciated that hydroxylamines may be accessed by reduction of oximes, for example by hydrogenation in the presence of a transition-metal containing catalyst or by a reducing agent such as sodium cyanoborohydride or sodium borohydride. Oximes may be prepared by condensation of a hydroxylamine with the corresponding aldehyde or ketone.

In one embodiment, Y includes from 1 to 25 carbon atoms, such as from 1 to 20, or from 1 to 15, for example from 1 to 10 carbon atoms. Y may include from 1 to 8 carbon atoms, such as from 1 to 5 carbon atoms. Y may include from 2 to 25 carbon atoms, such as from 3 to 25 carbon atoms, or from 2 to 20 carbon atoms, or from 2 to 15 carbon atoms.

### R²

As defined by formula (I), R² is a C1-30 group.

In one embodiment, R² is a C1-25 group, or a C1-20 group, such as a C1-15 group, or a C1-10 group. In one embodiment R² is a C1-8 group, or a C1-6 group, for example a C1-4 group. In one embodiment R² is a C1, C2, C3, C4 or C5 group. In one embodiment, R² is a C3-30 group, such as a C3-20 group, or a C3-10 group. It is preferred that R² is a C5-30 group, or a C6-30 group, such as a C6-14 group. R² may be a C3-8 group.

R² may comprise or consist of an alkyl group. The term “alkyl” may refer to a linear, branched and/or cyclic (“cycloalkyl”) hydrocarbon group that is saturated. Examples of alkyl groups include methyl, ethyl, n-propyl, isopropyl, n-butyl, isobutyl, sec-butyl, tert-butyl, n-pentyl, isopentyl, neopentyl or hexyl and the like. The term “cycloalkyl” refers to cyclic hydrocarbon groups. Examples of such groups include cyclopropyl, cyclobutyl, cyclopentyl, or cyclohexyl and the like. R² may comprise or consist of a C1-20 alkyl group, or a C1-15 alkyl group, or a C1-10 alkyl group. R² may comprise a C1-30 alkyl group, or a C3-20 alkyl group, or a C3-15 alkyl group; such as a C5-30 alkyl group, or a C5-20 alkyl group, or a C5-15 alkyl group. In one embodiment R² is a C1-30 alkyl group.

R² may comprise or consist of an alkenyl group. The term “alkenyl” may refer to a linear, cyclic and/or branched hydrocarbon group containing one or more carbon-carbon double bond. Examples of such groups include vinyl, allyl, prenyl, isoprenyl and the like. R² may comprise or consist of a C2-20 alkenyl group, or a C2-15 alkenyl group, or a C2-10 alkenyl group. R² may comprise a C3-30 alkenyl group, or a C3-20 alkenyl group, or a C3-15 alkenyl group; such as a C5-30 alkenyl group, or a C5-20 alkenyl group, or a C5-15 alkenyl group. In one embodiment R² is a C1-30 alkenyl group. R² may comprise a C2-6 alkenyl group, such as a C2-4 alkenyl group. R² may comprise a C2, C3, C4, C5 or C6 alkenyl group.

R² may comprise or consist of an alkynyl group. The term ‘alkynyl’ may refer to a linear, cyclic and/or branched hydrocarbon group containing one or more carbon-carbon triple bond. R² may comprise or consist of a C2-20 alkynyl group, or a C2-15 alkynyl group, or a C2-10 alkynyl group. R² may comprise a C3-30 alkynyl group, or a C3-20 alkynyl group, or a C3-15 alkynyl group; such as a C5-30 alkynyl group, or a C5-20 alkynyl group, or a C5-15 alkynyl group. In one embodiment R² is a C1-30 alkynyl group. R² may comprise a C2-6 alkynyl group, such as a C2-4 alkynyl group. R² may comprise a C2, C3, C4, C5 or C6 alkynyl group.

R² may comprise or consist of an aryl group. The term “aryl” may refer to carbocyclic aromatic groups including phenyl, naphthyl, anthracenyl, pyrenyl, chrysenyl, benz[a]anthracenyl, fluoranthene, indenyl, and tetrahydronaphthyl groups. For example, R² may comprise or consist of a C6-30 aryl group, or a C6-18 aryl group, preferably a C6-14 aryl group, for example a C6-10 aryl group. Preferably R² comprises a phenyl, naphthyl, anthracenyl, and/or pyrenyl group. For example, R² may comprise a phenyl, 2-naphthyl, 9-anthracenyl, and/or 1-pyrenyl group.

R² may comprise or consist of a heterocyclyl group. The term “heterocyclyl” may include aromatic (i.e. heteroaryl) and/or non-aromatic heteroatom-containing ring systems. Thus, for example, the term “heterocyclyl group” may include within its scope heterocyclyl ring systems that are aromatic, non-aromatic, unsaturated, partially saturated and/or fully saturated. Such groups may be monocyclic or polycyclic (e.g. bicyclic) and may contain, for example, 4 to 18 ring members, more usually 5 to 10 ring members, for example 5 or 6 ring members. R² may comprise or consist of a C5-30 heterocyclyl group, or a C5-18 heterocyclyl group, preferably a C5-14 heterocyclyl group, for example a C5-10 heterocyclyl group. Heterocyclyl groups may include 1, 2, 3 or 4 heteroatoms, more usually 1 or 2 or 3 heteroatoms, preferably 1 or 2 heteroatoms. Heterocyclyl groups may include heteroatoms selected from the list consistingof oxygen, nitrogen and sulfur.

A monocyclic group may include 3, 4, 5, 6, 7 or 8 ring members, more usually 4 to 7, and preferably 5, 6 or 7, more preferably 5 or 6 ring members. Examples of polycyclic (e.g. bicyclic) groups are those containing 6 to 18 ring members, more usually 8, 9 or 10 ring members. A saturated heterocyclic group may be selected from the list consisting of aziridine, azetidine, pyrrolidine, piperidine, piperazine, azepane, imidazolidine, tetrahydrofuran, oxirane, oxetane, oxane, oxepane, oxocane, 1,3-dioxolane, tetrahydrothiophene, pyrrolizidine, quinuclidine, 1-azaadamantane, 2-azaadamantane, 1-oxaspiro[4.5]decane, 1,4-dioxa-7-azaspiro[4.4.]nonane, decahydroisoquinoline and decahydroquinoline.

The heterocyclyl group may be a heteroaryl group having from 5 to 18 ring members, such as from 5 to 10 ring members, or even 5 or 6 ring members. Preferably R² comprises a heteroaryl group. The term “heteroaryl” is used herein to denote an aromatic heterocyclyl group. The term “heteroaryl” embraces polycyclic (e.g. bicyclic) ring systems wherein one or more rings are non-aromatic, provided that at least one ring is aromatic. In such polycyclic systems, the group may be attached by an aromatic ring, or by a non-aromatic ring. Examples of such polycyclic systems include 1,4,5,6-tetrahydrocyclopenta[b]pyrrole, indoline, tetrahydroquinoline, tetrahydroisoquinoline, 1,2-dihydroquinoline, 1,2-dihydroisoquinoline, 2H-benzo[e][1,3]oxazine, 2H-benzo[b][1,4]-oxazine, 2H-benzo[e][1,2]oxazine, 1H-isochromene and 2H-chromene. The heteroaryl group may be a five membered or six membered monocyclic ring or a bicyclic structure formed from fused five and six membered rings or two fused six membered rings. Preferably, the heteroaryl group contains one or two or more ring nitrogen atoms. Examples of heteroaryl groups include pyrrole, furan, thiophene, imidazole, furazan, oxazole, oxadiazole, oxatriazole, isoxazole, thiazole, thiadiazole, isothiazole, pyrazole, triazole, tetrazole, pyridine, pyrazine, pyridazine, pyrimidine, triazine, 1H-indole, 2H-isoindole, benzimidazole, 4-azaindole, 5-azaindole, 6-azaindole, 7-azaindole, benzofuran, isobenzofuran, benzo[c]thiophene, benzo[b]thiophene, benzo[d]isoxazole, benzo[d]thiazole, quinolone, isoquinoline, quinoxaline, phthalazine, quinazoline, cinnoline and 1,8-naphthyridine. The heteroaryl groups may include pyridine, imidazole and/or indole groups. Traditional numbering systems for these groups are shown below. In one embodiment, the R² group comprises a pyridine group bonded (directly or indirectly) to Y at the 2-, 3-, or 4-position of the pyridine ring. Preferably the R² group comprises a pyridine group bonded to Y at the 3-position of the pyridine ring. In one embodiment, the R² group comprises an imidazole group bonded to Y at the 5-position of the imidazole ring. In one embodiment, the R² group comprises an indole group bonded to Y at the 3-position of the indole ring.

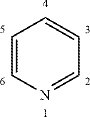

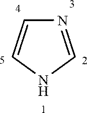

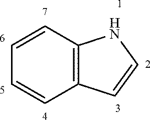

Preferably R² comprises or consists of a C5-30 aryl or heteroaryl group, such as a C5-20 aryl or heteroaryl group, or a C5-14 aryl or heteroaryl group.

In a preferred embodiment R² includes one or more group selected from the list consisting of pyridine, imidazole, indole, phenyl, naphthyl, anthracenyl, pyrenyl and isobutyl. More preferably R² includes one or more group selected from the list consisting of pyridine, imidazole, indole, phenyl, naphthyl, anthracenyl and pyrenyl.

The R² group may include one or two or three or more, such as from one to three, heteroatom-containing functional groups selected from the list consisting of amine (such as a primary, secondary or tertiary amine), amide, alcohol, ester, ether, thiol, thioether, sulfoxide, sulfone, sulfonamide, or halide (including fluorine, chlorine, bromine and iodine), dialkyl peroxide, disulfide, sulfonate ester, thioketone, thioester, phosphine, phosphonate ester, phosphate ester, boronic ester, borinic ester, borane, aldehyde, ketone, carbamate, carbonate, carboxylic acid, carboxylic acid anhydride, urea, ketal, acetal, orthoester, orthocarbonate, imine (e.g. an oxime and/or a Schiff base, such as a secondary ketamine and/or a secondary aldimine), imide, diimide, hydrazine, 1,2,3-triazole, hydrazone, acylhydrazone, semicarbazone, carbazate hydrazone, and hydrazone carboximidamide. In one embodiment R² includes an amine, especially where R² also includes a pyridine group.

The ratio of carbon atoms to heteroatoms in the R² group may be from 30:1 to 1:1, such as from 20:1 to 1:1, or from 10:1 to 1:1. The ratio of carbon atoms to heteroatoms in the R² group may be from 30:1 to 2:1, or from 30:1 to 4:1, or from 30:1 to 8:1.

In one embodiment the R² group may comprise a pyridine ring with a heteroatom-containing functional group. Preferably the heteroatom-containing functional group is in the 2-position of the pyridine ring. The pyridine ring may be bonded to Y at the 3-position of the pyridine ring. For example, the R² group may comprise a 3-(2-aminopyridine) group or a 2-pyridine group:

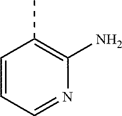

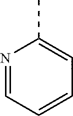

In a presently preferred embodiment, R² group is a 3-(2-aminopyridine) group and/or a 2-pyridine group.

In one embodiment, R² includes a functional group selected from the list consisting of:

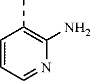

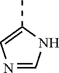

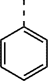

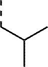

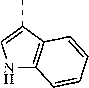

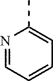

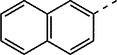

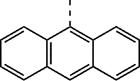

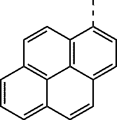

In one preferred embodiment formula (I) is as defined by any of the following formulae:

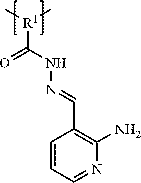

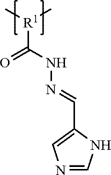

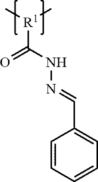

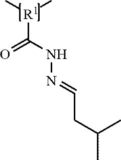

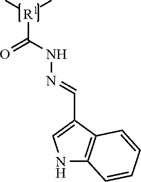

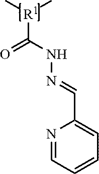

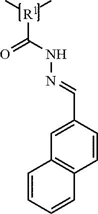

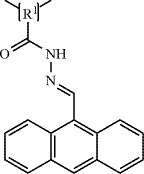

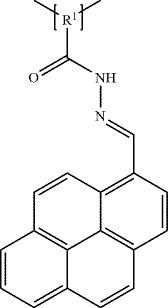

In a presently more preferred embodiment formula (I) is defined by any of the following formulae:

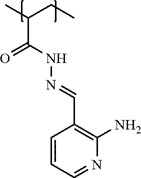

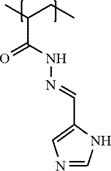

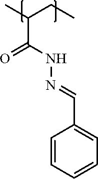

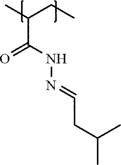

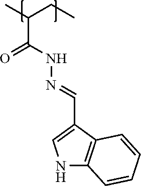

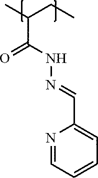

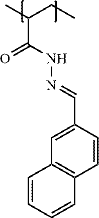

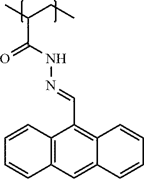

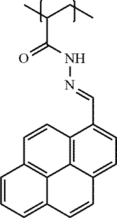

and compounds of the formula

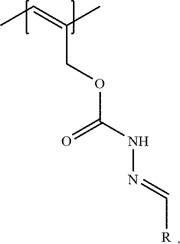

wherein the R groups are selected from the list consisting of:

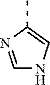

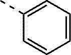

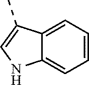

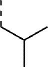

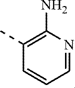

### Hydrophobicity

It will be appreciated that the surface characteristics of cells, for example charge and/or hydrophobicity, can differ between species of microorganisms, and may also differ between strains of the same species of microorganism. As such, whilst one R² group may be optimal for production of a microorganism-polymer complex for one microorganism, a different R² group may be optimal for another organism.

In one embodiment the R² group is hydrophobic, i.e. a hydrophobic C1-30 group. This may be particularly preferable for binding the polymer to cells with hydrophobic regions.

Hydrophobicity may be quantified by determining the distribution coefficient (logD) of the R² group. logD may be determined at a pH of 7.4 using n-octanol and an aqueous buffer solution. The logD of the R² group may be determined computationally to provide a clogD value. clogD may be calculated using the logD plugin in Marvin 17.6.0, 2017, ChemAxon. This may calculate clogD based on the method described in Viswanadhan, V. N. et al., J. Chem. Inf. Comput. Sci., 1989, 29, 163-172.

Hydrophobicity may be quantified by determining the partition coefficient (logP) of the R² group. The logP of the R² group may be determined computationally to provide a clogP value. clogP may be calculated using the logP plugin in Marvin 17.6.0, 2017, ChemAxon. This may calculate clogP based on the method described in Viswanadhan, V. N. et al., J. Chem. Inf. Comput. Sci., 1989, 29, 163-172.

It will be understood that the clogP and/or clogD of an R² group should be determined based on the R² group where an H atom has been used to replace the Y group.

The clogD and/or clogP of the R² group may be -2.0 or higher, or -1.5 or higher, preferably -1.0 or higher, such as -0.5 or higher, such as -0.2 or higher, for example 0.0 or higher, or 0.05 or higher. The clogD and/or clogP of the R² group may be 0.1 or higher, such as 0.15 or higher, or 0.2 or higher, or 0.25 or higher, or 0.5 or higher. The clogD and/or clogP of the R² group may be 1.0 or higher, such as 1.5 or higher. As shown by the examples, it has been found to be beneficial to use polymers with R² groups including such a clogD and/or clogP. The clogD and/or clogP may be 5.0 or higher, or even 10.0 or higher. The clogD and/or clogP of the R² group may be 13.0 or less, such as 10.0 or less. Preferably the clogD and/or clogP is 5.0 or less, for example 4.5 or less, or 4.0 or less. In one embodiment the clogD and/or clogP of the R² group is 3.5 or less, such as 3.0 or less, or 2.5 or less. This may allow the polymer to be sufficiently soluble to dissolve in the aqueous medium without requiring a water-miscible organic solvent, such as DMSO or acetic acid, to aid solubility. The clogD and/or clogP of the R² group may be from -0.5 to 5.0, or from 0.0 to 5.0, such as from 0.0 to 4.0. Preferably the clogD and/or clogP of the R² group is from -1.0 to 3.0, or from 0.5 to 2.5. In one embodiment the clogD and/clogP is from 1.0 to 5.0 or from 1.0 to 3.0.

It will be understood that heteroatom-containing functional groups may alter the hydrophobicity of the R² group at certain pH values. Preferably the R² group has three or fewer heteroatom-containing functional groups. However, the skilled person will be able to control the functionality of the R² group as described herein to ensure that the polymer remains suitably hydrophobic.

### Cleavable Polymers

The polymer may be cleavable. This may provide the benefit that the microorganism-polymer complex can be dispersed when desired. This may allow for a reaction performed by a microorganism in the microorganism-polymer complex to be controlled. For example, R¹ may be cleavable. The R² group may be cleavable from the R¹ group. Preferably the Y group is cleavable. More preferably, the Y group is cleavable into a first region, which is attached to the R¹ group, and a second region, which is attached to the R² group.

Cleavage may be performed by a variety of different means. For example, cleavage may be performed by hydrolysis, enzymes, in enzyme-mediated cleavage (such as of peptides, lipids or esters), light, in light-mediated cleavage (e.g. employing light-responsive chemical moieties such as nitro-benzene derivatives), change of pH, in pH-mediated cleavage (e.g. employingpH -cleavable chemical moieties such as acetal derivatives), and/or a competing agent, using dynamic covalent chemistries that can be cleaved in the presence of such competing agents (e.g. imines such as Schiff-bases). Such means have previously been described for different applications, for example by, and in the citations of, Leriche, G. et al., (2012) Bioorganic & Medicinal Chemistry, 20(2), 571-582 and Bargh, J. et al., (2019) Chemical Society Reviews, 48(16), 4361-4374. Preferably cleavage is performed by hydrolysis.

The imine of the Y group may be cleavable. The polymer may include one or more functional group that is cleavable in addition to the imine of the Y group. For example, the R¹ group and/or the Y group may include one or more functional group that is cleavable in addition to the imine of the Y group. A functional group that is cleavable may be selected from the list consisting of: nitrobenzene, amide, ester, amine (such as a secondary or tertiary amine), ether, dialkyl peroxide, thioether, disulfide, sulfoxide, sulfone, sulfonamide, sulfonate ester, thioketone, thioester, phosphine, phosphonate ester, phosphate ester, boronic ester, borinic ester, borane, ketone, carbamate, carbonate, carboxylic acid anhydride, urea, ketal, acetal, orthoester, orthocarbonate, imide, diimide, hydrazine, 1,2,3-triazole, hydrazone, acylhydrazone, semicarbazone, carbazate hydrazone, and hydrazone carboximidamide. Imines are C═N group containing moieties, such as Schiff bases (for example secondary ketamines and/or a secondary aldimines), oximes, hydrazones (e.g. alkyl, aryl and acyl hydrazones, semicarbazones, carbazate hydrazones, and hydrazone carboximidamides), or the structures shown in FIG. 1. These may be cleavable by hydrolysis. More preferably Y comprises a hydrazone, oxime, semicarbazone or carbazate hydrazone.

Preferably the polymer (e.g. the R¹ group and/or the Y group) is cleavable by hydrolysis. The polymer may be cleavable by hydrolysis under acidic or basic conditions. Such hydrolysis may be mediated by an additive such as a Brønsted acid, a Brønsted base, a Lewis acid, or a Lewis base. Brønsted acids may include perchloric acid, sulfuric acid, HI, HBr, HCl, HF, nitric acid, hydrogen sulfates, phosphoric acid, nitrous acid, acetic acid, carbonic acid and ammonium salts. Brønsted bases may include sulfate salts, dihydrogen phosphate salts, fluoride salts, nitride salts, acetate salts, hydrogen carbonate salts, hydrogen sulfate salts, ammonia, cyanide salts, carbonate salts, and hydroxide salts. Lewis acids may include a borane (e.g. borane or trimethylborane), TiCl₄, BF₃, SnCl₄, Et₃Al₂Cl₃ and AlCl₃. Lewis bases may include amines, phosphines, ethers, thioethers, selenoethers, DMAP, and pyridine. Preferably the polymer is cleavable by hydrolysis mediated by a Brønsted acid or a Brønsted base, more preferably by a Brønsted acid. Hydrolysis is preferably performed in the presence of water.

A Y group including an imine, such as a hydrazone or another group disclosed in FIG. 1, may be cleavable. The C═N moiety, for example of a hydrazone or an acyl hydrazone, may be cleavable under acidic and/or basic conditions. As such, hydrolysis may be mediated by an additive such as a Brønsted acid, a Brønsted base, a Lewis acid or a Lewis base, preferably by a Brønsted acid. Hydrolysis of imines such as hydrazones may be mediated by change of pH and/or by addition of a competing agent.

A cleavable functional group used in the polymer of the present invention may be cleavable at a pH of 5 or lower, such as 4 or lower, or 3 or lower, or 2 or lower, or 1 or lower. A cleavable functional group used in the polymer of the present invention may be cleavable at a pH of 9 or higher, such as 10 or higher, or 11 or higher, or 12 or higher, or 13 or higher.

### Microorganism

The present invention can be employed with any microorganism. The microorganism used may naturally perform strongly or weakly at forming biofilms. The microorganism used may not naturally be able to form a biofilm. The microorganism may be a prokaryotic or an eukaryotic microorganism. A prokaryotic microorganism may be from the domain of bacteria or archaea. Examples of suitable eukaryotic microorganisms may include any microorganism from the kingdom Protista, Plantae, Fungi, or Animalia. Preferably the microorganism is a bacteria or a fungi. More preferably the microorganism is a bacteria.

Particularly preferred microorganisms include those which can produce a chemical and/or biological product, e.g. a medicament, such as an organic molecule, or a protein binding fragment, such as an antibody fragment, a bulk chemical and/or fine chemical. Suitable fungi may include a yeast or a mold. For example, the microorganism may be in a genera selected from the list consisting of Saccharomyces (e.g. Saccharomyces cerevisiae), Schizosaccharomyces (e.g. Schizosaccharomyces pombe), Candida (e.g. Candida albicans), Neurospora (e.g. Neurospora crassa), Aspergillus (e.g. Aspergillus nidulans), Paraconiothyrium, Alternaria and Phomopsis.

Suitable fungi may include a fungi from the division of Ascomycota, such as from the order of Hypocreales, or from the family of Hypocreaceae, for example from the genus of Acremonium. The cephalosporins, a class of β-lactam antibiotics, were derived from Acremonium fungi. Therefore, new ways of facilitating the formation of biofilm-like structures of such organisms may be particularly commercially useful. Suitable fungi from the division of Ascomycota may also include fungi from the class of Eurotiomycetes, such as from the order of Eurotiales, for example from the family of Trichocomaceae, such as a fungi from the genus of Penicillium. Species of the genus Penicillium produce penicillin, a common antibiotic. Therefore, new ways of facilitating the formation of biofilm-like structures of such organisms may be particularly commercially useful.

Examples of suitable archaea may include any microorganism from the phyla of Euryarchaeota or Crenarchaeota. Suitable Archaea may include any microorganism in a Genera selected from the list consisting of Thermococcus (e.g. Thermococcus litoralis), Sulfolobus, Archaeoglobus (e.g. Archaeoglobus fulgidus), Haloferax, Haloarcula, Halococcus, Natronococcus, Halobacterium, Natrialba, Haloterrigena, Haloquadratum, Halorubrum, Natronobacterium, Natronococcus, Methanococcus and Methanopyrus.

Suitable bacteria may include any microorganism in a phyla selected from the list consisting of Acidobacteria, Actinobacteria, Aquificae, Armatimonadetes, Bacteroidetes, Caldiserica, Chlamydiae, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae and Verrucomicrobia. Preferably the microorganism is in the phylum of Proteobacteria.

Suitable bacteria within the phylum of Actinobacteria may include any microorganism in the order of Actinomycetales, such as in the family of Pseudonocardiaceae, for example within the genus of Amycolatopsis. Within the genus of Amycolatopsis, suitable bacteria include A. orientalis, from which the antibiotic vancomycin can be isolated. Therefore, new ways of facilitating the formation of biofilm-like structures of such organisms may be particularly commercially useful. Suitable bacteria within the phylum of Actinobacteria may include any microorganism in the class of Actinomycetes, such as within the order of Actinomycetales, for example within the family of Pseudonocardiaceae, for instance within the genus of Saccharopolyspora. Within the genus of Saccharopolyspora, suitable bacteria include S. erythraea, which produces the macrolide antibiotic erythromycin. Therefore, new ways of facilitating the formation of biofilm-like structures of such organisms may be particularly commercially useful. Suitable bacteria within the phylum of Actinobacteria may include any microorganism within the family of actinomycetaceae. Such a microorganism may be within the genus of Streptomyces. Bacteria within the genus of Streptomyces naturally produce antifungals, antiparasitics and antibiotics, such as streptomycin, neomycin, cypemycin, grisemycin, bottromycins and chloramphenicol. Therefore, new ways of facilitating the formation of biofilm-like structures of such organisms may be particularly commercially useful. Suitable bacteria within the Streptomyces genus may include any species selected from the list consisting of S. noursei, S. nodosus, S. natalensis, S. venezuelae, S. roseosporus, S. fradiae, S. lincolnensis, S. alboniger, S. griseus, S. rimosus, S. aureofaciens, S. antibioticus, S. torulosus, Streptomyces sp. SPB74, S. bambergiensis, S. ghanaensis, S. clavuligerus, Streptomyces sp. K01-0509, S. avermitilis, S. platensis, S. verticillus, S. staurosporeus, S. hygroscopicus, and S. viridochromogenes.

Suitable bacteria within the phylum of proteobacteria may include any microorganism in a class selected from the list consisting of Alphaproteobacteria, Betaproteobacteria, Hydrogenophilalia, Gammaproteobacteria, Acidithiobacillia, Deltaproteobacteria, Epsilonproteobacteria, and Oligoflexia. Preferably the microorganism is in the class of Gammaproteobacteria. Suitable bacteria within the class of Gammaproteobacteria may include any microorganism in an order selected from the list consisting of Xanthomonadales, Chromatiales, Cardiobacteriales, Legionellales, Ruthia, Vesicomyosocius, Thiomicrospira, Thiotrichales, Oceanospirillales, Alteromonadales, Aeromonadales, Vibrionales, Pasteurellales, Pseudomonadales and Enterobacteriales. Preferably the microorganism is in the order of Enterobacteriales or Pseudomonadales. Suitable bacteria may include any microorganism within the order of Vibrionales. For example, suitable bacteria may be in the order of Vibrionales and within a genus selected from the list consisting of Aliivibrio, Allomonas, Beneckea, Enhydrobacter, Listonella, Lucibacterium, Photobacterium, Salinivibrio and Vibrio. Within the Vibrio genus, suitable bacteria may include any species selected from the list consisting of V. adaptatus, V. aerogenes, V. aestivus, V. aestuarianus, V. agarivorans, V. albensis, V. alfacsensis, V. alginolyticus, V. anguillarum, V. areninigrae, V. artabrorum, V. atlanticus, V. atypicus, V. azureus, V. brasiliensis, V. bubulus, V. calviensis, V. campbellii, V. casei, V. chagasii, V. cholera, V. cincinnatiensis, V. coralliilyticus, V. crassostreae, V. cyclitrophicus, V. diabolicus, V. diazotrophicus, V. ezurae, V. fluvialis, V. fortis, V. furnissii, V. gallicus, V. gazogenes, V. gigantis, V. halioticoli, V. harveyi, V. hepatarius, V. hippocampi, V. hispanicus, V. ichthyoenteri, V. indicus, V. kanaloae V. lentus V. litoralis, V. logei, V. mediterranei, V. metschnikovii, V. mimicus, V. mytili V. natriegens, V. navarrensis, V. neonates, V. neptunius, V. nereis, V. nigripulchritudo, V. ordalii, V. orientalis, V. pacinii, V. parahaemolyticus, V. pectenicida, V. penaeicida, V. pomeroyi, V. ponticus, V. proteolyticus, V. rotiferianus, V. ruber, V. rumoiensis, V. salmonicida, V. scophthalmi, V. splendidus, V. superstes, V. tapetis, V. tasmaniensis, V. tubiashii, V. vulnificus, V. wodanis and V. xuii. The bacteria may be V. cholera.

Suitable bacteria may include any microorganism in a family selected from the list consisting of Moraxellaceae, Oceanospirillaceae, Pseudomonadaceae, Pseudoalteromonadaceae, Idiomarinaceae, Shewanellaceae and Psychromonadaceae. Suitable bacteria may include a microorganism in the order of Enterobacteriales and in the family of Enterobacteriaceae. Suitable bacteria may be from the family of Enterobacteriaceae and in a genus selected from the list consisting of Alterococcus, Arsenophonus, Biostraticola, Brenneria, Buchnera, Budvicia, Buttiauxella, Cedecea, Citrobacter, Cosenzaea, Cronobacter, Dickeya, Edwardsiella, Enterobacillus, Enterobacter, Erwinia, Escherichia, Ewingella, Franconibacter, Gibbsiella, Hafnia, Izhakiella, Kosakonia, Klebsiella, Kluyvera, Leclercia, Lelliottia, Leminorella, Levinea, Lonsdalea, Mangrovibacter, Moellerella, Morganella, Obesumbacterium, Pantoea, Pectobacterium, Phaseolibacter, Photorhabdus, Phytobacter, Plesiomonas, Pluralibacter, Pragia, Proteus, Providencia, Pseudocitrobacter, Rahnella, Raoultella, Rosenbergiella, Rouxiella, Saccharobacter, Salmonella, Samsonia, Serratia, Shigella, Shimwellia, Siccibacter, Sodalis, Tatumella, Thorsellia, Trabulsiella, Wigglesworthia, Xenorhabdus, Yersinia, and Yokenella. Examples of suitable bacteria may include any microorganism in a genus selected from the list consisting of Escherichia (e.g. Escherichia coli), Alteromonas (e.g. Alteromonas hispanica), Salipiger (e.g. Salipiger mucosus), Palleronia (e.g. Palleronia marisminoris), Idiomarina, Methylobacterium, Staphylococcus, Micrococcus, Rhodococcus, Ralstonia, Paracoccus, Magnetospirillum and Methylococcus (e.g. Methylococcus capsulatus). Presently preferred bacteria include those of the genus Escherichia, especially E. coli.

Suitable Bacteria may include any microorganism within the order of Pseudomonadales, preferably within the family of Pseudomonadaceae, more preferably within the genus of Pseudomonas. Suitable bacteria within the genus of Pseudomonas may include any species selected from the list consisting of P. aeruginosa, P. alcaligenes, P. anguilliseptica, P. argentinensis, P. borbori, P. citronellolis, P. flavescens, P. mendocina, P. nitroreducens, P. oleovorans, P. pseudoalcaligenes, P. resinovorans, P. straminea, P. asplenii, P. aurantiaca, P. aureofaciens, P. chlororaphis, P. corrugate, P. fragi, P. lundensis, P. taetrolens, P. antarctica, P. azotoformans, P. blatchfordae, P. brassicacearum, P. brenneri, P. cedrina, P. corrugate, P. fluorescens, P. gessardii, P. libanensis, P. mandelii, P. marginalis, P. mediterranea, P. meridian, P. migulae, P. mucidolens, P. orientalis, P. panacis, P. protegens, P. proteolytica, P. rhodesiae, P. synxantha, P. thivervalensis, P. tolaasii, P. veronii, P. denitrificans, P. pertucinogena, P. cremoricolorata, P. entomophila, P. fulva, P. monteilii, P. mosselii, P. oryzihabitans, P. parafulva, P. plecoglossicida ,P. putida, P. balearica, P. luteola, P. stutzeri, P. amygdali, P. avellanae, P. caricapapayae, P. cichorii, P. coronafaciens, P. ficuserectae, P. helianthi, P. meliae, P. savastanoi, P. syringae, P. tomato, P. viridiflava, P. abietaniphila, P. acidophila, P. agarici, P. alcaliphila, P. alkanolytica, P. amyloderamosa, P. asplenii, P. azotifigens, P. cannabina, P. coenobios, P. congelans, P. costantinii, P. cruciviae, P. delhiensis, P. excibis, P. extremorientalis, P. frederiksbergensis, P. fuscovaginae, P. gelidicola, P. grimontii, P. indica, P. jessenii, P. jinjuensis, P. kilonensis, P. knackmussii, P. koreensis, P. lini, P. lutea, P. moraviensis, P. otitidis, P. pachastrellae, P. palleroniana, P. papaveris, P. peli, P. perolens, P. poae, P. pohangensis, P. protegens, P. psychrophila, P. psychrotolerans, P. rathonis, P. reptilivora, P. resiniphila, P. rhizosphaerae, P. rubescens, P. salomonii, P. segitis, P. septica ,P. simiae, P. suis, P. teessidea, P. thermotolerans, P. toyotomiensis, P. tremae, P. trivialis, P. turbinellae, P. tuticorinensis, P. umsongensis, P. vancouverensis, P. vranovensis and P. xanthomarina; in particular P. putida, P. fluorescens and P. aeruginosa.

### Method

The first aspect provides a method of producing a microorganism-polymer complex. The method comprises the step of exposing, in an aqueous medium, cells of a microorganism to a polymer comprising groups as defined by formula (I).

### Cell Preparation

The method may include a step, before the cells of the microorganism are exposed to the polymer, of incubating the microorganism in an aqueous medium containing a growth medium.

The growth medium may comprise or consist of solutes to sustain the life of the microorganism. The skilled person will understand that different growth media are suited to different microorganisms. Suitable growth media may include Luria-Bertani (LB) broth and/or M63 media (https://www.protocolsonline.com/molecular-biology/media-molecular-biology/m63-medium/; accessed 10 Oct. 2019). Preferably the growth medium is, or includes, LB broth. LB broth includes tryptone, yeast extract and sodium chloride in amounts of 10 g, 5 g and 10 g per litre respectively, in water. LB broth typically consists of a solution of these compounds that has been autoclaved, for example at 121° C. for 20 minutes, and cooled or allowed to cool. The growth medium may comprise 25% or more LB broth, such as 35% or more, or 45% or more LB broth by volume. The growth medium may comprise from 25% to 95% LB broth, such as from 25% to 75% LB broth, or from 35% to 65% LB broth by volume. It will be understood that different microorganisms may perform preferentially under different conditions. The growth medium may include tryptone in an amount of from 1 to 100 g per litre, for example from 5 to 50 g per litre, such as from 5 to 15 g per litre. The growth medium may include yeast extract in an amount of from 0.5 to 100 g per litre, for example from 1 to 50 g per litre, such as from 1 to 10 g per litre. The growth medium may include sodium chloride in an amount of from 1 to 100 g per litre, for example from 5 to 50 g per litre, such as from 5 to 15 g per litre. The growth medium may include tryptone, yeast extract and sodium chloride. This may be particularly preferred where the microorganism is, or is related to, E. coli.

M63 media may be described as a minimal, low osmolarity media for microorganisms such as E. coli. M63 media typically includes 2 g (NH₄)₂SO₄, 13.6 g KH₂PO₄ and 0.5 mg FeSO₄·7H₂O in 1 litre of deionised water, and the resulting solution is typically adjusted to pH 7 with KOH. M63 media may optionally include 1 ml 1 M MgSO₄.7H₂O, 10 ml 20% carbon source (e.g. a sugar, such as glucose, or glycerol), 0.1 ml 0.5% vitamin B1 (thiamine), 5 ml 20% casamino acids or L-amino acids to 40 µg/ml or DL-amino acids to 80 µg/ml, and antibiotic. The growth medium may comprise 25% or more M63 media, such as 35% or more, or 45% or more M63 media by volume. The growth medium may comprise from 25% to 95% M63 media, such as from 25% to 75% M63 media, or from 35% to 65% M63 media by volume. It will be understood that different microorganisms may perform preferentially under different conditions. The growth medium may include (NH₄)₂SO₄ in an amount of from 0.1 to 100 g per litre, such as from 1 to 50 g per litre, for example from 5 to 20 g per litre. The growth medium may include KH₂PO₄ in an amount of from 1 to 500 g per litre, for example from 10 to 200 g per litre, such as from 40 to 100 g per litre. The growth medium may include FeSO₄·7H₂O in an amount of from 0.1 to 200 mg per litre, for example from 0.5 to 50 mg per litre, such as from 1 to 10 mg per litre. The growth medium may include (NH₄)₂SO₄, KH₂PO₄ and FeSO₄·7H₂O.

The step of incubating the microorganism in an aqueous medium containing a growth medium may be performed at a temperature of from 5° C. to 60° C., such as from 10° C. to 50° C., or from 15° C. to 40° C. Preferably the temperature is from 20° C. to 40° C., such as from 25° C. to 35° C.

The microorganism may be incubated in the aqueous medium containing a growth medium for a period of 3 hours or more, such as 6 hours or more. Preferably the microorganism is incubated for a period of 12 hours or more, such as 18 hours or more. The period may be 72 hours or less, such as 48 hours or less, or 24 hours or less. For example, the period may be from 3 hours to 72 hours, such as from 12 to 24 hours. The microorganism may be incubated in the aqueous medium containing a growth medium until the resulting culture reaches an optical density (OD, e.g. at 600 nm) of 0.10 or more, such as 0.12 or more, or 0.15 or more, such as 0.18 or more, for example 0.19 or more, or 0.20 or more. The amount of the aqueous medium (e.g. buffer) may be controlled to give an OD of 0.5 or less, such as 0.45 or less, or 0.40 or less, or 0.35 or less, such as 0.30 or less, or 0.25 or less. The OD may be from 0.10 to 0.40, such as from 0.15 to 0.35, or from 0.18 to 0.32, such as from 0.20 to 0.30, for example from 0.20 to 0.25. This may ensure that the bacteria are in the lag phase and/or has not yet reached the exponential stage of growth where they are adapting to the growth conditions. However, it may be that the bacteria are allowed to grow to reach an OD of 0.3 or above, such as 0.5 or above to achieve best yields. More aqueous medium containing a growth medium may be added to the microorganism if required to increase the OD to be within the desired range.

In one embodiment the cells are washed before they are exposed to the polymer. The cells may be washed with water, such as deionised water, a buffer and/or the media in which the cells will be exposed to the polymer.

In one embodiment the cells are isolated after being washed and before being exposed to the polymer. This may allow the aqueous medium to be swapped between the initial incubation and exposure to the polymer for optimum performance. For example, the cells may be isolated by centrifugation. Centrifugation may be performed at 4000 g of force or more, such as from 4000 g to 20000 g of force. The cells may be washed and then isolated once or twice or more.

### Exposing Cells of a Microorganism to the Polymer

Cells of a microorganism are exposed, in an aqueous medium, to a polymer comprising groups as defined by formula (I).

The method of the first aspect may include the step of suspending and/or dissolving, the polymer and/or the cells in the aqueous medium. The polymer and/or the cells may, therefore, initially, be in a dispersed state in the aqueous medium. This may enable the microorganism-polymer complex to form more efficiently and/or be produced in a suspended and/or dissolved state in the water so as to increase the surface area of the microorganism-polymer complex beyond that where, for example, the microorganism-polymer complex is solely attached to the walls of a vessel.

The polymer may be added to the microorganism, or the microorganism may be added to the polymer. The microorganism and the polymer may be added to the aqueous medium simultaneously (e.g. the aqueous medium is added to a mixture of the polymer and the microorganism, or a mixture of the polymer and the microorganism is added to the aqueous medium), sequentially (i.e. microorganism before polymer or polymer before microorganism), or separately (e.g. the microorganism may be mixed with a first medium and the polymer may be mixed with a second medium, and the first and second media may be combined.

The microorganism and the polymer may each be provided in solid state (e.g. as a powder) or in a liquid state (for example as a solution or dispersion (e.g. suspension) in a water-miscible solvent, which may comprise water, dimethylsulfoxide (DMSO), N,N-dimethylformamide (DMF), N,N-dimethylacetamide (DMAC), THF, dioxane (e.g. 1,4-dioxane) and/or acetic acid). Water-miscible solvents other than water may be helpful to increase the solubility of the polymer in the aqueous medium. It will be understood that the water-miscible solvent should not be toxic to the microorganism.

Preferably the polymer should be at least partially dispersible, for example partially or fully soluble, in the aqueous medium. It will be understood that the aqueous medium should be a liquid. The polymer may be provided in a form that is dispersible in the aqueous medium, for example as a powder, granules, pellets and/or flakes, or as a solution in a water-miscible solvent, such as dimethyl sulfoxide (DMSO).

The polymer and/or the cells may be mixed (e.g. stirred, swirled, shaken or subjected to ultrasonication) in the aqueous medium. The polymer and/or the cells may be mixed with one another.

In one embodiment the cells are suspended in an amount of the aqueous medium (e.g. a buffer, for example a potassium phosphate buffer, preferably with a concentration of from 0.08 M to 0.12 M) before they are exposed to the polymer. It will be understood that the higher the concentration of cells, the higher the optical density (OD) is. The amount of the aqueous medium (e.g. buffer) may be controlled to give an OD of 0.10 or more, such as 0.12 or more, or 0.15 or more, such as 0.18 or more, for example 0.19 or more, or 0.20 or more. The amount of the aqueous medium (e.g. buffer) may be controlled to give an OD of 0.5 or less, such as 0.45 or less, or 0.40 or less, or 0.35 or less, such as 0.30 or less, or 0.25 or less. The OD may be from 0.10 to 0.40, such as from 0.15 to 0.35, or from 0.18 to 0.32, such as from 0.20 to 0.30, for example from 0.20 to 0.25. This may ensure that the bacteria are in the lag phase and/or has not yet reached the exponential stage of growth where they are adapting to the growth conditions. However, it may be that the bacteria are allowed to grow to reach an OD of 0.3 or above, such as 0.5 or above to achieve best yields.

In one embodiment, the cells are exposed to the polymer by adding the polymer to a suspension of the cells in the aqueous medium. It will be understood that even very small amounts of the polymer may enable the microorganism-polymer complex to be produced.

The concentration of the polymer in the aqueous medium, for example after adding the buffer and the growth medium, may be 0.0001 mg/mL or more, or 0.001 mg/mL or more, such as 0.005 mg/mL or more. Preferably the concentration is 0.01 mg/mL or more, such as 0.02 mg/mL or more, or 0.04 mg/mL or more, for example 0.05 mg/mL or more. In one embodiment the concentration is from 0.0001 mg/mL to 5.00 mg/mL, such as from 0.001 mg/mL to 1.00 mg/mL, or from 0.005 mg/mL to 0.50 mg/mL. Preferably the concentration is from 0.01 mg/mL to 0.10 mg/mL, such as from 0.02 mg/mL to 0.08 mg/mL, or 0.04 mg/mL to 0.07 mg/mL, for example from 0.05 mg/mL to 0.06 mg/mL.

In one embodiment a growth medium, such as M63 media, is added to the aqueous medium after the polymer has been added. The skilled person will understand that different growth media are suited to different microorganisms. Preferably the aqueous medium includes the growth medium, such as M63 media, and the buffer. The amount of the growth medium in relation to the buffer may be 10% or more, such as 25% or more, or 40% or more by volume. Preferably the amount of the growth medium in relation to the buffer is 50% or more, such as 60% or more, or 75% or more, or 85% or more by volume. For example, the amount of the growth medium in relation to the buffer may be from 10% to 500%, such as from 25% to 400%, or from 40% to 300% by volume. Preferably the amount of the growth medium in relation to the buffer is from 50% to 250%, such as from 60% to 200%, or from 75% to 150%, or from 85% to 125% by volume.

The aqueous medium should be suitable for survival of the microorganism. It will be understood that the aqueous medium should not be toxic to the microorganism. The aqueous medium includes water. The water employed in the methods of the present invention may be deionised water, distilled water or tap water. The aqueous medium may comprise 80% or more water by weight, such as 90% or more, or 95% or more water by weight. The aqueous medium may have a pH of from 2 to 12, such as from 4 to 10, or from 5 to 9, or from 6 to 8. It will be understood that some microorganisms tolerate particularly basic and/or acidic conditions. The microorganism-polymer complex may reduce the natural pH sensitivity of a microorganism. For example, the pH may be 0 or more, such as 1 or more, or 14 or less, such as 11 or less, such as from 0 to 14, or from 1 to 11.

The aqueous medium may include or consist of a buffer, such as a phosphate-based buffer. Preferably the buffer is a buffer of KH₂PO₄ and K₂HPO₄ (i.e. a potassium phosphate buffer). The concentration of the buffer (before being added to the aqueous medium) may be from 0.01 M to 1.0 M, or from 0.05 M to 0.5 M, preferably from 0.08 M to 0.12 M, for example about 0.1 M. The aqueous medium (for example, the composition of the aqueous medium after all components have been added for the formation of the complex, but excluding the volume of the cells and the polymer for this calculation) may comprise 25% or more of the buffer, such as 35% or more, preferably 45% or more of the buffer by volume. The aqueous medium may comprise from 25% to 95% of the buffer, such as from 25% to 75% of the buffer, or from 35% to 65% of the buffer, preferably from 45% to 55% of the buffer by volume.

The aqueous medium may include or consist of a culture (e.g. growth) medium such as LB broth or M63 media, as discussed above. The aqueous medium may comprise 25% or more of the culture medium, such as 35% or more, or 45% or more of the culture medium by volume. The aqueous medium may comprise from 25% to 95% of the culture medium, such as from 25% to 75%, or from 35% to 65% of the culture medium by volume.

The aqueous medium may include one or more water-miscible solvent selected from the list consisting of DMSO, DMF, DMAC, THF, dioxane (e.g. 1,4-dioxane) and acetic acid. Such water-miscible solvents may be helpful to increase the solubility of the polymer in the aqueous medium. Preferably the aqueous medium includes DMSO. Preferably the polymer is soluble in the water-miscible solvent. The water-miscible solvent may be included in the aqueous medium in an amount of 50% or less, such as 40% or less, or 25% or less, such as 10% or less by volume. For example, water-miscible solvent may be included in the aqueous medium in an amount of from 1% to 50%, or from 2% to 40%, such as from 5% to 30% by volume.

The aqueous medium may include an antibiotic, such as ampicillin, tetracycline, kanamycin and/or chloramphenicol. The antibiotic (e.g. ampicillin) may be included in an amount of 10 µg mL⁻¹ or more, such as 100 µg mL⁻¹.

The cells may be exposed to the polymer at a temperature of from 5° C. to 60° C., such as from 10° C. to 50° C., or from 15° C. to 40° C. In one embodiment the temperature is from 15° C. to 35° C., such as from 15° C. to 30° C., or from 15° C. to 25° C. In one embodiment the temperature is from 25° C. to 50° C., such as from 30° C. to 45° C., or from 35° C. to 40° C. The temperature may be from 20° C. to 40° C., such as from 25° C. to 30° C. For example, the step of exposing, in an aqueous medium, cells of a microorganism to a polymer comprising a group as defined by formula (I) may be performed at such a temperature.

The microorganism-polymer complex may be produced under aerobic or under anaerobic conditions.

It will be understood that, especially where the polymer is cleavable, the conditions under which the microorganism-polymer complex is produced should not cleave the polymer. The skilled person will be able to determine, using the common general knowledge, conditions under which the polymer may cleave and avoid these. The microorganism-polymer complex may be formed under conditions that do not hydrolyse the polymer.

The microorganism-polymer complex may be produced in substantially neutral pH conditions to prevent cleavage (e.g. hydrolysis) of any pH cleavable, such as imine-containing, moieties present. For example, the pH of the aqueous medium whilst the microorganism and the polymer are exposed to each other may be substantially neutral. For example, the pH may be 2.0 or higher, or 3.0 or higher, such as 4.0 or higher, or 5.0 or higher. In one embodiment pH is 6.0 or higher, such as 6.5 or higher, or 7.0 or higher. The pH may be 14.0 or lower, or 12.0 or lower, such as 10.0 or lower, or 8.0 or lower. The pH may be from 3.0 to 14.0, such as from 4.0 to 12.0, or from 4.0 to 10.0. Preferably the pH is from 4.0 to 9.0, such as from 5.0 to 8.0, or from 6.0 to 7.5, for example about 7.0. The microorganism-polymer complex may be produced in the absence of (e.g. the aqueous medium may not include) a Lewis acid or Lewis base such as those listed in the Cleavable polymers section above.

In one embodiment the method of the first aspect may be performed in a vessel. The method may include adding the polymer, the microorganism and/or the aqueous medium (or components of the aqueous medium) to the vessel. The vessel may be formed of glass. The vessel may have a volume of 500 mL or more, such as 1 L or more, or 5 L or more, or 50 L or more.

The microorganism and the polymer may be exposed to each other for a period of time sufficient for the microorganism-polymer complex to be produced. of 1 minute or more, such as 30 minutes or more, or 1 hour or more, such as 6 hours or more. The period of time may be sufficient for the production of microorganism-polymer complex to have substantially completed, such as for the microorganism-polymer complex to have reached 80% completion or more, such as 90% completion or more, or 95% completion or more. Preferably the period of time is 12 hours or more, such as 18 hours or more, or 24 hours or more, or 36 hours or more. For example, the period of time may be from 1 minute to 2 weeks, such as from 30 minutes to 1 week (7 days), or from 1 hour to 5 days, or from 6 hours to 4 days. In one embodiment the period of time is from 12 hours to 4 days, such as from 18 hours to 3 days, or from 24 hours to 48 hours.

The microorganism and the polymer may be mixed with one another in the aqueous medium. This may aid interaction of the microorganism and the polymer, and thereby may aid formation of the microorganism-polymer complex. Mixing may be performed using an orbital shaker. The speed of mixing may be from 10 rpm to 1000 rpm, such as from 20 rpm to 500 rpm or from 50 rpm to 250 rpm. The microorganism and the polymer may be mixed in an orbital shaker at about 150 rpm with a throw of about 19 mm.

### Microorganism-Polymer Complex

The method defined by the first aspect of the invention produces a microorganism-polymer complex. The second aspect defines a microorganism-polymer complex. The complex comprises cells of a microorganism and a polymer comprising groups as defined by formula (I). In one embodiment the microorganism-polymer complex is produced by (obtainable by) the method of the first aspect. The complex of the second aspect of the invention may be obtained by the method of the first aspect.

It will be understood that, in the complex, cells of the microorganism will have aggregated onto the polymer. As such, there may be some attraction between the cells and the polymer that holds these species together. Such attraction may be caused by a non-covalent interaction. The microorganism-polymer complex may appear like and/or behave like a naturally formed biofilm. Cells of the microorganism may aggregate onto/around the complex to form a biofilm.

The complex may be two-dimensional (e.g. sheet-like) or three dimensional.

### Detection of the Microorganism-Polymer Complex

The presence of a complex of the polymer with the microorganism, as opposed to a simple mixture of the polymer and the microorganism, may be determined by various techniques.

For example, the microorganism-polymer complex may be detected and measured by laser diffraction, e.g. using a Malvern Mastersizer 2000 or a Coulter LS230 particle size analyser (Beckman Coulter, High Wycombe, UK). It will be understood that use of such equipment can include adding sample of the culture to a flow cell filled with H₂O (<14 mL) under moderate stirring (default speed 5 setting) to the required concentration as indicated by the in-built display software (obscuration of 8-12%). Particle size ranges can be defined with reference to PSS-Duke standards (Polymer Standard Service, Kromatek Ltd, Dunmow, UK). The size of complexes in the culture can then be measured. Complex size distribution can be determined as a function of the complex diffraction, for example using the Coulter software (version 2.11a) and plotted as a function of the percentage of distribution volume.

An individual microorganism-polymer complex may have a size (e.g. diameter) of 3.0 µm or more, such as 4.0 µm or more, or 5.0 µm or more. The size of an individual complex may be 10 µm or more, such as 20 µm or more, or 40 µm or more. For example, the size of an individual complex may be 100 µm or more, such as 200 µm or more, or 500 µm or more. In one embodiment, the size is from 3.0 µm to 1 cm, such as from 4.0 µm to 5 mm, or from 5.0 µm to 1 mm.

The microorganism-polymer complexes in a culture may have a number average (e.g. mean) size (e.g. diameter) of 3.0 µm or more, such as 4.0 µm or more, or 5.0 µm or more. The average size may be 10 µm or more, such as 20 µm or more, or 40 µm or more. For example, the average size may be 100 µm or more, such as 200 µm or more, or 500 µm or more. In one embodiment, the average size is from 3.0 µm to 1 cm, such as from 4.0 µm to 5 mm, or from 5.0 µm to 1 mm.

The presence of a microorganism-polymer complex may be determined by an increase in the number average (e.g. mean) size (e.g. diameter) of particles in a culture with a polymer according to formula (I), compared to a control culture without the polymer. For example, the increase in average size may be 5% or more, such as 10% or more, or 50% or more, or 100% or more, or 200% or more, or 500% or more compared to the control culture. The average size in the test sample may be from 5% to 2000% more than that of the control culture, such as from 10% to 1000%, or from 50% to 750%.

The presence of a microorganism-polymer complex according to the invention may be determined by crystal violet staining. For example, crystal violet staining may be performed by first washing the complex/cells with deionised water. Washing may be facilitated by the flocculation of the cells with the polymer, which makes the complex precipitate and may allow the supernatant to be removed relatively easily, to isolate the complex. Centrifugation may be used to assist isolation of the complex. The washed and isolated complex is stained with 1% crystal violet solution for 1 h. The stained mixture is washed three times with deionised water and isolated, as described above, to remove excess crystal violet. Acetic acid (33% in deionised water) is added to the isolated stained complex in an amount sufficient to just dissolve the crystal violet. Then the absorbance of the solution at 550 nm is measured using a plate reader, such as a BMG CLARIOstar Plus plate reader.

The absorbance (at 550 nm) of a crystal violet-stained sample of a culture including the microorganism-polymer complex according to the invention (test sample) may be more than the absorbance of a sample of the crystal violet-stained culture taken immediately after the cells are exposed to the polymer in the aqueous medium (comparative sample, e.g. the culture including the cells, the polymer, the growth medium and the buffer). The absorbance of the test sample may be 1% or more higher than that of the comparative sample, such as 10% or more, or 20% or more, or 50% or more higher. Preferably the absorbance of the test sample is 100% or more higher than the absorbance of the comparative sample, such as 200% or more, or 500% or more, or 750% or more higher than the absorbance of the comparative sample. The absorbance of the test sample may be from 1% to 2000% of the absorbance of the comparative sample, such as from 10% to 1000%, or from 50% to 750%.

Where a microorganism has been engineered to express green fluorescent protein (GFP) in an amount corresponding to the amount of expression of a protein relating to biofilm formation (e.g. Curli protein), the presence of a microorganism-polymer complex according to the invention may be determined by measuring the fluorescence of the GFP. Fluorescence may be measured using a plate reader, such as a BMG CLARIOstar Plus plate reader. The excitation wavelength may be 488 nm and the emission wavelength may be 510 nm. For example, the ratio of fluorescence of a culture including a microorganism-polymer complex according to the invention to that of a culture produced in the same manner but without the polymer of formula (I) may be greater than 1:1. In other words, the fluorescence of the culture containing the microorganism-polymer complex according to the invention may be higher than that of the culture produced in the same manner but without the polymer of formula (I). The ratio may be 1.05:1 or more, or 1.1:1 or more, such as 1.2:1 or more, or 1.5:1 or more, or 2:1 or more.

The presence of a microorganism-polymer complex may be determined at any suitable time following the exposure of the cells of the microorganism to the polymer. For example, the presence of a microorganism-polymer complex may be determined at 1 hour after exposure, or 2 hours or 24 hours or 3 days, or 5 days after exposure.

The presence of a microorganism-polymer complex may be determined by staining biofilmassociated polysaccharides, for example using the methods described by Burton, E., et al., J Ind. Microbiol. Biotechnol. (2007) 34:1.

The presence of a microorganism-polymer complex may be determined visually, for example by scanning electron microscopy (SEM).

The presence of a microorganism-polymer complex according to the invention may be determined by turbidimetry. As measured by turbidimetry, the optical density (OD, for example at 600 nm) of a culture including the microorganism-polymer complex according to the invention (test sample) may be more than the OD of the culture immediately after the cells are exposed to the polymer in the aqueous medium (comparative sample, e.g. the culture including the cells, the polymer, the growth medium and the buffer). The OD of the test sample may be 1% or more higher than the OD of the comparative sample, such as 10% or more, or 20% or more, or 50% or more higher. Preferably the OD of test sample is 100% or more higher than the OD of the comparative sample, such as 200% or more, or 500% or more, or 750% or more higher than the OD of the OD of the comparative sample. The OD of the test sample may be from 1% to 2000% of the OD of the comparative sample, such as from 10% to 1000%, or from 50% to 750%.

Depending on the OD (for example at 600 nm) of the culture immediately after the cells are exposed to the polymer in the aqueous medium, a culture including the microorganism-polymer complex according to the invention may have an OD (for example at 600 nm) of 0.01 or more, such as 0.05 or more, for example 0.1 or more, or 0.15 or more. Preferably the OD of a culture including the microorganism-polymer complex according to the invention is 0.20 or more, such as 0.25 or more, or 0.30 or more. For example, the OD of a culture including the microorganism-polymer complex according to the invention may be 0.50 or more, such as 0.70 or more or 1.0 or more. The OD of a culture including the microorganism-polymer complex according to the invention may be from 0.01 to 4.0, such as from 0.1 to 2.0, for example 0.1 to 1.5. Preferably the OD of a culture including the microorganism-polymer complex according to the invention is from 0.20 to 1.5, such as from 0.20 to 1.25, or from 0.20 to 1.0. It will be understood that the microorganism-polymer complex may sediment from the culture. Therefore, it may be possible for the culture including the microorganism-polymer complex according to the invention to have an OD equal to or less than the OD of the culture immediately after the cells are exposed to the polymer in the aqueous medium. The formation of a sediment after exposing the cells to the polymer may be indicative of the production of the complex.

Therefore, the presence of the microorganism-polymer complex can be detected.

### Optional Steps After the Microorganism-Polymer Complex Has Been Produced

The method may include the step of separating the microorganism-polymer complex from the aqueous medium. For example, the microorganism-polymer complex may be separated from the aqueous medium by centrifugation. The method may include the step of drying, such as freeze drying (i.e. subjecting to lyophilisation) the microorganism-polymer complex, to remove water. Freeze drying is performed at a temperature of 0° C. or below, such as -15° C. or below, and under reduced pressure, such as 6 mbar or less, or 2 mbar or less. Conventional drying (by evaporation) may be performed at a temperature such as at 10° C. or more, or 20° C. or more, or 30° C. or more, and optionally under reduced pressure, such as 500 mbar or less, or 100 mbar or less, such as 10 mbar or less, or 2 mbar or less. Conventional drying may be performed under a flow of gas, such as air or nitrogen. Drying the microorganism-polymer complex provides dry microorganism-polymer complex, which may have a water content of 10% or less, such as 5% or less, or 2% or less, such as 1% or less by weight.

The microorganism-polymer complex may be stored. As such, the method may include storing the microorganism-polymer complex. The microorganism-polymer complex may be stored at 20° C. or lower, such as 5° C. or lower, or 0° C. or lower, such as -10° C. or lower, for example -50° C. or lower. The microorganism-polymer complex may be stored for a period of time. For example, the microorganism-polymer complex may be stored for one day or more. The microorganism-polymer complex may be stored for two days or more, or three days or more, or a week or more, such as a month or more.

### Dispersing the Complex

The present inventors have additionally determined that it may be beneficial to be able to disperse a microorganism-polymer complex that has been formed. For example, this may be useful when controlling the rate at which a reaction proceeds, for controlling the rate of growth of the complex and/or for controlling the rate of production of a desired product. This may be useful when terminating a reaction performed with the microorganism-polymer complex. This may be useful when cleaning apparatus used to perform such a reaction.

According to the fifth aspect, the present invention provides a method of dispersing a microorganism-polymer complex, wherein the microorganism-polymer complex is defined by the second aspect, wherein the polymer is cleavable, and wherein the method comprises the step of cleaving the polymer.

It will be understood that cleaving the polymer will reduce the cohesion between cells. This may allow the cells to fall away from one another, naturally and/or with agitation e.g. stirring, allowing the microorganism to change into a planktonic form. Thus, a reaction performed by the microorganism-polymer complex may be controlled.

For example, the method may include the step of cleaving the R¹ group, the R² group and/or the Y group. The R² group may be cleaved from the R¹ group. Preferably the method includes the step of cleaving the Y group. The Y group may be cleavable into a first region, which is attached to the R¹ group, and a second region, which is attached to the R² group.

Cleavage may be performed by a variety of different means. For example, cleavage may be performed by hydrolysis, enzymes, in enzyme-mediated cleavage (such as of peptides, lipids or esters), light, in light-mediated cleavage (e.g. employing light-responsive chemical moieties such as nitro-benzene derivatives), change of pH, in pH-mediated cleavage (e.g. employing pHcleavable chemical moieties such as acetal derivatives), and/or a competing agent, using dynamic covalent chemistries that can be cleaved in the presence of such competing agents (e.g. imines such as Schiff-bases). Such means have previously been described for different applications, for example by, and in the citations of, Leriche, G. et al., (2012) Bioorganic & Medicinal Chemistry, 20(2), 571-582 and Bargh, J. et al., (2019) Chemical Society Reviews, 48(16), 4361-4374. Preferably cleavage is performed by hydrolysis.

To cleave the polymer (e.g. the R¹ group, the R² group and/or the Y group), the complex may be hydrolysed, subjected to one or more enzymes (e.g. protease and/or lipase), subjected to light (e.g. visible and/or UV light), subjected to a change in pH, and/or subjected to a competing agent.

Hydrolysis may be preferable as the conditions for hydrolysis may be controlled to allow the polymer to re-form after it has been dispersed. Hydrolysis may be performed under acidic or basic conditions. As such, hydrolysis may be mediated by an additive such as a Brønsted acid, a Brønsted base, a Lewis acid or a Lewis base, for example those described in the Cleavable polymers section above. Hydrolysis is preferably performed in the presence of water.

The polymer of the microorganism-polymer complex may be hydrolysed (cleaved) under nonneutral (i.e. acidic or basic) pH conditions. For example, the polymer may be cleaved under an acidic pH of 6 or lower, preferably 5 or lower, or more preferably 4 or lower. The polymer may alternatively be cleaved under a basic pH of 8 or higher, preferably 9 or higher, or more preferably 10 or higher. It may be preferable to not use very acidic or basic conditions to cleave the polymer so that, for example, the integrity of the microorganism is maintained. For example, the polymer may be cleaved at a pH of 1 or higher, preferably from 2 to 6, or more preferably from 3 to 5. Alternatively, the polymer may be cleaved at a pH of 14 or lower, preferably from 8 to 13, or more preferably from 9 to 12, such as from 9 to 11. Cleavage may be performed by modifying the pH and/or adding a competing agent, such as a primary acylhydrazine (hydrazide), for example at an acidic pH, e.g. from 2 to 7, for example from 4 to 6.5 (Park K.D. et al., Chemistry & Biology, Volume 16, Issue 7, Pages 763-772).

The polymer may be used to controllably form a microorganism-polymer complex. For example, the polymer may be used to form a microorganism-polymer complex, and then the complex may be dispersed, and then the complex may be reformed.

Hydrolysis may be performed in a buffer, such as a KH₂PO₄/K₂HPO₄ buffer, and/or a growth medium. Prior to hydrolysis, the pH of the buffer may be from 2 to 12, such as from 3 to 11, or from 4 to 10. The regions towards the endpoints of such ranges may be particularly suitable to extremophile organisms that are adapted to strongly acidic and/or basic conditions. Preferably the initial pH of the buffer is about 7, such as from 5 to 9, or from 6 to 8. The polymer will then be added to form the microorganism-polymer complex. To disperse the microorganism-polymer complex, the pH of the solution containing the microorganism-polymer complex may be adjusted. Where a KH₂PO₄/K₂HPO₄ buffer is used, this may adjust ratio of KH₂PO₄:K₂HPO₄ in situ (https://www.unl.edu/cahoonlab/phosphate%20buffer.pdf, accessed 9 Dec. 2019). For example, the pH may be made more acidic by adding (as a solution in an aqueous solvent or neat) a Brønsted acid. The Brønsted acid may include an inorganic Brønsted acid such as KH₂PO₄, HCl, H₂SO₄ and/or HNO₃, and/or an organic acid such as citric acid, carbonic acid tartaric acid so as to reduce the pH of the buffer and so as to hydrolyse the functional group, such as the imine. Preferably the pH may be made more alkaline by adding (as a solution in an aqueous solvent or neat) a Brønsted base, such as a potassium-based Brønsted base, for example KOH, K₂HPO₄ and/or K₂CO₃, so as to increase the pH of the buffer and so as to hydrolyse the functional group, such as the imine. The pH of the solution containing the microorganism-polymer complex may be adjusted to 3 or less, such as 2 or less, or 1 or less, such as 0 or less. The pH of the solution containing the microorganism-polymer complex may be adjusted to 11 or more, such as 12 or more, or 13 or more.

As shown by the examples, it has been found that the microorganism-polymer complex can be reformed after it has been dispersed.

### Applications of the Microorganism-Polymer Complex

### Chemical/Biological Transformations

It will be appreciated that the microorganism-polymer complex may be used to perform a chemical/biological transformation, for example in the field of chemical (e.g. pharmaceutical, fine chemical or bulk chemical) synthesis.

The present invention provides, according to a third aspect, a method for producing a chemical and/or biological product, the method comprising exposing a substrate to a microorganism-polymer complex as defined by the second aspect. According to a fourth aspect, the present invention provides a chemical and/or biological product obtainable by (produced by) the third aspect.

It will be appreciated that the skilled person is aware of a vast number of chemical transformations that can be performed (e.g. catalysed) by microorganisms and biofilms thereof. The exact chemical transformation performed, substrate used, or product produced may, therefore, not particularly limited. The present invention may enable the skilled person to perform a chemical transformation on a commercial scale that was not previously able to be performed at such scale because the microorganism was not able to form a biofilm-like structure such as the microorganism-polymer complex.

Furthermore, the skilled person is aware of a vast number of biotransformations/biosyntheses that can be performed by various microorganisms and biofilms thereof. These may be performed by the microorganism-polymer complex of the present invention. For example, the complex may be used for biosynthesis, for example to synthesise antibiotics, vitamins, steroids, alkaloids, amino acids and pharmaceutical enzymes (Therapeutic Use of Medicinal Plants and Their Extracts: Volume 1, Springer, Cham, ISBN 978-3-319-63861-4, pp 105-123). The exact biosynthesis performed is, therefore, not particularly limited. The present invention may enable the skilled person to perform a biosynthesis on a commercial scale that was not previously able to be performed at such scale because the microorganism was not able to form a biofilm-like structure such as the microorganism-biofilm complex.

In one embodiment the chemical and/or biological product is a medicament, such as an active pharmaceutical ingredient, or an intermediate thereof. The medicament may be an organic molecule of molecular weight up to 5000 g mol⁻¹, such as up to 2000 g mol⁻¹, or up to 1000 g mol⁻¹. The chemical and/or biological product may be an organic molecule, a protein binding fragment and/or an antibody. The chemical and/or biological product may be a fine chemical and/or a bulk chemical.

In one embodiment of the third and/or fifth aspects, the microorganism-polymer complex is provided by performing the method of the first aspect. The methods of the third and/or fifth aspects may include the method of producing a microorganism-polymer complex of the first aspect. The methods of the third and/or fifth aspects may include embodiments disclosed in relation to the first aspect. The method of the fifth aspect may include the method for producing a chemical and/or biological product of the third aspect. The method of the fifth aspect may include embodiments disclosed in relation to the method of the third aspect.

The substrate is exposed to the microorganism-polymer complex to produce the chemical and/or biological product. For example, the chemical and/or biological product may be provided by performing a chemical transformation and/or biosynthesis. In other words, the substrate may react with the microorganism-polymer complex, a component and/or a discharge thereof. The substrate may be exposed to the microorganism-polymer complex (e.g. a reaction may be performed) in a vessel. The substrate may be exposed to the microorganism-polymer complex in an aqueous reaction medium. The aqueous reaction medium should be suitable for survival of the microorganism in the complex. It will be understood that the aqueous reaction medium should not be toxic to the microorganism in the complex. The aqueous reaction medium includes water. The water employed in the methods of the present invention may be deionised water, distilled water or tap water. The aqueous reaction medium may comprise 60% or more water by weight, such as 80% or more, or 90% or more, such as 95% or more water by weight. The pH of the aqueous reaction medium may be 0 or more, such as 1 or more, and/or 14 or less, such as 11 or less. The aqueous reaction medium may have a pH of from 2 to 12, such as from 4 to 10, or from 5 to 9, or from 6 to 8.

The aqueous reaction medium may include or consist of a buffer such as a phosphate-based buffer. Preferably the buffer is a buffer of KH₂PO₄ and K₂HPO₄ (i.e. a potassium phosphate buffer). The concentration of the buffer (before being added to the aqueous reaction medium) may be from 0.01 M to 1.0 M, or from 0.05 M to 0.5 M, preferably from 0.08 M to 0.12 M, for example about 0.1 M. The aqueous reaction medium may comprise 25% or more of the buffer, such as 35% or more, such as 45% or more of the buffer by volume. The aqueous reaction medium may include 60% or more of the buffer, such as 70% or more, or 80% or more. The aqueous reaction medium may comprise from 25% to 95% of the buffer, such as from 25% to 75% of the buffer, or from 35% to 65% of the buffer, preferably from 45% to 55% of the buffer by volume. The buffer may have a pH of from 4.0 to 10.0, such as from 5.0 to 9.0. It may be preferred that the buffer has a pH of from 6.0 to 8.0, such as from 6.5 to 7.5.

The aqueous reaction medium may include or consist of a growth medium such as LB broth or M63 media, as discussed above. The aqueous reaction medium may comprise 25% or more of the growth medium, such as 35% or more, or 45% or more of the growth medium by volume. The aqueous reaction medium may comprise from 25% to 95% of the growth medium, such as from 25% to 75%, or from 35% to 65% of the growth medium by volume.

The aqueous reaction medium may include one or more water-miscible solvent selected from the list consisting of DMSO, DMF, DMAC, THF, dioxane (e.g. 1,4-dioxane) and acetic acid. Such water-miscible solvents may be helpful to increase the solubility of the substrate(s) and/or the reagent(s) in the aqueous reaction medium. Preferably the aqueous reaction medium includes DMSO. Preferably the polymer is soluble in the water-miscible solvent. The water-miscible solvent may be included in the aqueous reaction medium in an amount of 50% or less, such as 40% or less, or 25% or less, such as 10% or less by volume. For example, water-miscible solvent may be included in the aqueous reaction medium in an amount of from 1% to 50%, or from 2% to 40%, such as from 5% to 30% by volume. In particular, the aqueous reaction medium may include DMSO in an amount of from 0.1% to 30% by volume. For example, the amount may be from 0.5% to 20%, or from 1% to 15% by volume. Preferably the amount is from 2% to 10%, such as from 4% to 6%, by volume. The aqueous reaction medium may include an antibiotic, such as ampicillin. The antibiotic (e.g. ampicillin) may be included in an amount of 10 µg mL⁻¹ or more, such as 100 µg mL⁻¹.

The microorganism-polymer complex may be provided in any suitable form, for example as a solid (e.g. as a powder), or as a liquid (e.g. as a dispersion and/or a solution in the aqueous reaction medium, such as water or a buffer, or DMSO).

One or more substrates may be exposed to the microorganism-polymer complex. It will be understood that the substance including the substrate, the microorganism-polymer complex defines a reaction mixture and the aqueous reaction medium. The reaction mixture may include one or more substrates. The substrate(s) may be mixed with the microorganism-polymer complex. The substrate(s) may be provided in any suitable form, for example as a solid (e.g. as a powder), or as a liquid (e.g. as a neat liquid and/or as a dispersion and/or as a solution in an aqueous reaction medium, such as water or a buffer, or DMSO).

During the reaction the concentration of the microorganism-polymer complex may be adjusted such that the OD (for example at 600 nm) of the reaction mixture (i.e. the volume including the complex and the aqueous reaction medium), is 0.01 or more, such as 0.05 or more, for example 0.1 or more, or 0.15 or more. The OD of the reaction mixture may be 0.20 or more, such as 0.25 or more, or 0.30 or more. For example, the OD of the reaction mixture may be 0.50 or more, such as 0.70 or more or 1.0 or more. The OD of the reaction mixture may be from 0.01 to 4.0, such as from 0.1 to 2.0, for example 0.1 to 1.5. The OD of the reaction mixture may be from 0.20 to 1.5, such as from 0.20 to 1.25, or from 0.20 to 1.0. The concentration of the complex may be adjusted by controlling the amount of the aqueous reaction medium added to the complex.

The concentration of the substrate in the aqueous reaction medium may be 0.1 mM or more, such as 0.5 mM or more, preferably 1 mM or more. The concentration may be 100 mM or less, such as 50 mM or less, or 20 mM or less, preferably 10 mM or less, such as 5 mM or less. The concentration may be from 0.1 mM to 100 mM, such as from 0.5 mM to 50 mM, preferably from 1 mM to 10 mM.

As noted above, it will be appreciated that the skilled person is aware of a vast number of chemical transformations and biosyntheses that can be performed (e.g. catalysed) by microorganisms and biofilms thereof. The substrate may depend upon the desired chemical transformation or biosynthesis. By way of example, the substrate could be a haloindole, such as a 5-haloindole. This family of compounds may include 5-fluoroindole, 5-chloroindole and 5-bromoindole. A haloindole can be chemically transformed by cells of a microorganism into a halotryptophan, such as a 5-halotryptophan - an important class of pharmaceutical intermediates.

E. coli may be engineered to perform the transformation of haloindole to halotryptophan by a recombinant tryptophan synthase TrpBA expressed constitutively from plasmid pSTB7 (Tsoligkas, A.N. et al., (2011) Chembiochem 12:1391-1395; Tsoligkas A.N. et al., (2012) Colloids Surf B: Biointerfaces 89:152-160; Kawasaki, H. et al., (1987) J Biol Chem 262:10678-10683).

In another example, a microorganism, such as E. coli, may be used to hydrolyse an ester, such as 4-nitrophenyl dodecanoate.

One or more reagents (e.g. cofactor and/or coenzyme) may be exposed to, such as mixed with, the substrate and the microorganism-polymer complex. Reagent(s) may be provided in any suitable form, for example as a solid (e.g. as a powder), or as a liquid (e.g. as a neat liquid and/or as a dispersion and/or as a solution, for example in a water-miscible solvent). The amount and/or concentration of each reagent may depend upon its role in the specific transformation or biosynthesis taking place.

The product of the chemical transformation or biosynthesis, such as halotryptophan, may be separated from the microorganism-polymer complex, the substrate, the reagent, or preferably the reaction mixture as a whole. The product may be separated from the microorganism-polymer complex, the substrate, and/or any reagent, preferably the reaction mixture as a whole, using liquid/liquid and/or solid/liquid separation techniques. For example, the product may be separated from the reaction mixture using one or more techniques selected from the list consisting of filtration, chromatography, crystallisation, evaporation, freeze-drying and/or distillation.

### Agricultural Applications

The methods and complexes of the claimed invention may find application in the agricultural field. For example, a complex may be coated upon, or be formed upon, an article. The article may be made of a plastic, a glass, a metal, a natural material (e.g. wood, or a leaf, stem, trunk or root of a plant, such as a crop) or be a live object. For example, the article may be a seed.

Alternatively, a complex may be applied to soil, such as by spraying (e.g. as described by Ciccillo et al., Environ. Microbiol., 2002 Apr;4(4):238-45). It will be understood that the exact nature of soil changes according to location. The soil may be suitable for growing crops.

The complex may provide the coated article, such as the seed, or soil with properties derived from the microorganism. For example, the complex may provide enhanced disease resistance, more rapid germination, and/or enhanced growth characteristics to the seed, for example through the delivery of active compounds such as plant growth regulators, micronutrients, and microbial inoculants, which may be integrated within or produced by the complex. The complex may improve appearance and handling characteristics of the article (e.g., seed weight and size) or the soil. The microorganism being in the form of the complex may make the microorganism more robust than if the microorganism had been applied to the article, such as the seed, or soil in another form.

The seeds to be coated may include the seeds of legumes (i.e. plants in the family Fabaceae (or Leguminosae)), such as soybeans (Glycinemax).

For agricultural applications such as seed coating or soil treatment, the microorganism incorporated into the complex may be Rhizobia, Frankia and/or Trichoderma, bacterial endophytes that can colonize some or a portion of a plant’s interior tissues, and cyanobacteria. Other suitable microorganisms for these applications may include Agrobacteria, such as Agrobacterium radiobacter, Azospirillum , such as Azospirillum brasilense, Azospirillum lipoferum, Azotobacter, such as Azotobacter chroococcum, Bacillus, such as Bacillus fimus, Bacillus licheniformis, Bacillus megaterium, Bacillus mucilaginous, Bacillus pumilus, Bacillus spp., Bacillus subtilis, Bacillus subtilis var. amyloliquefaciens, Burkholderia, such as Burkholderia cepacia, Delfitia, such as Delfitia acidovorans, Paenobacillus, such as Paenobacillus macerans, Pantoea, such as Pantoea agglomerans, Pseudomonas, such as Pseudomonas aureofaciens, Pseudomonas chlororaphis, Pseudomonas fluorescens, Pseudomonas solanacearum, Pseudomonas spp., Pseudomonas syringae, Serratia, such as Serratia entomophilia, Streptomyces, such as Streptomyces griseoviridis, Streptomyces spp., Streptomyces lydicus and Rhizobia spp. Such bacteria may be termed plant-growth promoting bactieria (PGPB).

The main types of seed coatings include seed dressing, film coating, and pelleting, which can be chosen according to the purpose of application and the type of seed or selected microbes. The complex may be applied to the article, such as the seed, with a binder and, optionally, a filler that can act as a carrier. The seed may be coated with the microorganism by spraying the complex (which may be dispersed in a solvent, such as water) onto the seed, or immersing the seeds in a dispersion of the complex (e.g. as described by Ciccillo et al., Environ. Microbiol., 2002 Apr;4(4):238-45). The coating may be provided on one or more surface of the article. The coating may be substantially continuous across the surface(s), or around the article, such as the seed.

### Probiotics

The complexes may be used to sustain or promote growth of microorganisms that are beneficial to the gut. The methods and complexes of the invention may be applied to probiotic microorganisms. For example, probiotic microorganisms may be of a genus selected from the list consisting of Lactobacillus, Bifidobacterium, Saccharomyces, Streptococcus, Enterococcus, Escherichia, and Bacillus. Complexes including such microorganisms may be used to benefit, for example improve or maintain, gastrointestinal health, for example the gut microbiome.

For such applications the complex may be administered orally. A composition for oral administration may be formulated as a pill, e.g. a tablet or capsule, lozenge, thin film, a solution or suspension (e.g., drink or syrup), a powder, liquid or solid crystals, natural or herbal plant, seed, or food of sorts (preferably as a yoghurt or milk-based drink), or as a paste. Such compositions may be administered to a human or animal.

### Skincare

The complexes may be used to sustain or promote growth of microorganisms that are beneficial to the skin. Complexes may find application in the field of skincare. Microorganisms beneficial for skincare applications may include microorganisms selected from the list consisting of: Bifidobacterium, Lactobacillus, Vitreoscilla, Bacillus coagulans, Propionibacterium and Staphylococci, such as Staphylococcus hominis, Staphylococcus epidermidis, and Streptococcus thermophilus. For example, the complexes may find application in the reduction of skin sensitivity, treatment of acne, treatment of eczema, treatment of dry skin, reduction of ageing.

For skincare applications the complex may be formulated for topical application, for example as a cream, ointment, paste, lotion or gel. Skincare compositions may be administered (e.g. applied) to a human or animal.

### Oral Hygiene

The complexes may be used to sustain or promote growth of microorganisms that are beneficial to the buccal cavity (mouth). Microorganisms beneficial for oral hygiene applications can include microorganisms selected from the list consisting of: Streptococci, such as Streptococcus mitis. Streptococcus sanguinis. Streptococcus gordonii, Streptococcus salivarius K1 2, Streptococcus dentisani and Streptococcus A12 , Lactobacilli, and Bifidobacterium. The complexes may aid oral hygiene or health, for example by reducing halitosis (bad breath) or gingivitis.

For oral administration a complex may be formulated as a pill, e.g. a tablet (such as buccal, sub-lingual, or orally-disintegrating tablet) or capsule, lozenge, thin (buccal) film, a solution or suspension (e.g., gargle, drink or syrup), a powder, liquid or solid crystals, natural or herbal plant, seed, or food of sorts (preferably as a yoghurt or milk-based drink), or as a paste (e.g., toothpaste). For oral hygiene applications, the composition is preferably formulated as a lozenge, buccal, sub-lingual, or orally disintegrating tablet, solution or suspension, for example as a gargle, or as a paste. Compositions for oral hygiene may be administered to a human or animal.

### BRIEF DESCRIPTION OF THE DRAWINGS

The invention will be further illustrated in a non-limiting manner by the accompanying drawings, in which:

FIG. 1 shows imine-containing functional groups that may be used as part of the linker group Y;

FIG. 2 shows the results of laser diffraction analysis comparing the size distribution (by %volume) of complexes induced by polymer pAH-2AFP and biofilms formed without the polymer after 48 h of incubation;

FIG. 3 shows the absorbance of cultures incubated for 24 hours (bottom graph) or 48 hours (top graph) with polymer pAH or a polymer according to the invention, following staining with crystal violet;

FIG. 4 shows the green fluorescent protein (GFP) fluorescence of cultures over 48 hours’ incubation with or without polymer pAH-2AFP;

FIG. 5 shows the green fluorescent protein (GFP) fluorescence of cultures over 48 hours’ incubation with or without a polymer containing a carbazate hydrazone linker;

FIG. 6 shows the normalised GFP expression at 48 hours for cultures with or without polymer pAH-2AFP;

FIG. 7 shows, for cultures with polymer pAH or polymers according to the invention, the fold change in GFP fluorescence, compared to a control culture without polymer;

FIG. 8 shows, for cultures with polymers containing carbazate hydrazone linkers, the change in GFP fluorescence, compared to a control culture without polymer;

FIG. 9 shows, for cultures with or without polymer pAH-2AFP, the effect of pH change on crystal violet absorbance;

FIG. 10 shows the percentage conversion of 5-fluoroindole to 5-flurotryptophan in cultures with polymer pAH or polymers according to the invention after 48 hours, correlated with the clogD for the R² group of that polymer; and

FIG. 11 shows the kinetic trace for the hydrolysis of 4-nitrophenyl dodecanoate to 4-nitrophenol in cultures with or without polymer pAH-Bn or polymer pAH-In.

### EXAMPLES

### Synthesis of Polymers

### Materials and Methods

Chemicals were purchased from Sigma-AldrichO® , Fisher Scientific®, VWR® or AcrosO® , and were used without further purification. All solvents were reagent grade or above, purchased from Sigma-AldrichO® , Fisher ScientificO® or VWRO® , and were used without further purification.

Nuclear Magnetic Resonance (NMR) spectra were recorded on either a Bruker Avance III 300 MHz, a Bruker Avance III 400 MHz spectrometer, a Varian Mercury 300 MHz or a Varian Inova 500 MHz spectrometer. Chemical shifts are reported in ppm (δ units) referenced to the following solvent signals: DMSO-d₆ δH 2.50, D₂O δH 4.79 and CDC1₃, δH 7.26. Infrared (IR) spectra were recorded on a Perkin Elmer Spectrum Two FT-IR spectrometer. Ultraviolet-visible (UV-vis) spectra were recorded on a Campsec M550 Double Beam Scanning UV-vis Spectrophotometer or a Cary 50 Spectrophotometer. Size Exclusion Chromatography (SEC), AKA Gel Permeation Chromatography (GPC) spectra were recorded on a Shimadzu Prominence LC-20A fitted with a Thermo Fisher Refractomax 521 Detector or a SPD20A UV-vis Detector. In all cases flow rate was 1 mL min⁻¹. Reactive polymers were analysed using Dulbecco’s Phosphate Buffered Saline 0.0095 M (PO₄) without Ca and Mg as the eluent and a flow rate of 1 mL min⁻¹. The instrument was fitted with an Agilent PL aquagel-OH column (300 × 7.5 mm, 8 mm) and run at 35° C. Molecular weights were calculated based on a standard calibration method using polyethyleneglycol standards. Protected polymers were analysed using 0.05 M LiBr in dimethylformamide (DMF) at 60° C. as the eluent. The instrument was fitted with a Polymer Labs PolarGel guard column (50 × 7.5 mm, 5 µm) followed by two PLGel PL1110-6540 columns (300 × 7.5 mm, 5 µm). Alternatively, molecular weights were calculated based on a standard calibration method using polymethylmethacrylate standards. Dialysis was carried out in deionised water at room temperature for a minimum of 48 hours using a Spectra/Por 6 1000 Molecular weight cut-off (MWCO) 38 mm width membrane.

### Synthesis of Polymers

Synthesis of Poly(acryloyl hydrazide) pAHx

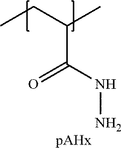

The synthesis of pAHx is described in Crisan D.N. et al., Polym. Chem., 2017, 8, 4576-4584 (where pAHx is described as “Px”) and in the supporting information relating to Angew. Chem. Int. Ed. 2016, 55, 7492-7495; DOI: 10.1002/anie0.201601441; https://onlinelibrary.wiley.com/action/downloadSupplement?doi=10.1002%2Fanie0.201601441 &file=anie201601441-sup-0001-misc₋information. pdf, accessed 2 Sep. 2019 (where pAHx is described as “P1”).

It will be understood that the “x” of pAHx signifies the approximate number of monomer units of the polymer. The end groups of the pAH polymer have been shown as methyl groups for simplicity only.

### Calculation of DP Using ¹H-NMR

The degree of polymerisation (DP) in pAHx was calculated from the ¹H-NMR spectra by comparing the integration of the methyl substituents in the 2-(2-methylpropionic acid) end-group (0.95 and 1.01 ppm, 6 H) to the integration from the aliphatic region in the polymer backbone (1.59-2.08 ppm) (as shown in Figure S2 and Table S1 of the supporting information relating to Angew. Chem. Int. Ed. 2016, 55, 7492-7495; DOI: 10.1002/anie0.201601441; https://onlinelibrary.wiley.com/action/downloadSupplement?doi=10. 1002%2Fanie0.201601441&file=anie201601441-sup-0001-misc_information.pdf, accessed 2 Sep. 2019).

DP may alternatively be calculated by dividing the polymer mass by the monomer mass, where polymer mass can be determined from the decrease in integration of the vinylic proton peaks of the monomer (6.2 and 5.6 ppm, 3 H) as observed by ¹H NMR.

### Synthesis of Poly(acetylene)s

### Synthesis of Acetylene Monomers

O-propargyl-N-(tert-butoxycarbonyl)amino carbamate PBocAC

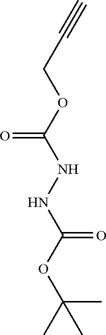

Propargyl Alcohol (259 mg, 4.67 mmol) was dissolved in EtOAc (10 ml). Carbonyldiimidazole (665 mg, 5.13 mmol) was dissolved in EtOAc (10 ml). The two solutions were combined and the mixture was stirred at 60° C. for 2 hours. tert-Butyl carbazate (925 mg, 7.00 mmol) was dissolved into EtOAc (10 ml). The resulting solution was added to the previous crude, and the mixture was allowed to react for a further 2 hours. The organic layer was concentrated under reduced pressure, then suspended again in EtOAc (20 ml). The organic layer was washed with deionized H₂O (9 × 20 ml) and 0.1 M HC1 (3 × 20 ml). The solvent was then removed on a rotary evaporator and to give the target compound as white powder (500 mg, 47% yield). ¹H NMR (400 MHz, CDC13) /ppm; 6.52 (s, 1H, H-N), 6.30 (s, 1H, H-N), 4.79 (d, 2H, J= 2.5 Hz, H—C═), 2.52 (t, 1H, J= 2.5 Hz, H—CΞ), 1.50 (s, 9H, H-C). ¹³C NMR (400 MHz, CDC13) /ppm; 155.69 (OC(O)N), 81.87 (CC═C), 75.15 (C═CH), 53.32 (C2CO), 27.93 (CCH3). FTIR vmax /cm¹; 3279 (s, sh, H—C═), 3024 (w, sh, H-N), 2979 (w, sh, H-C), 2136 (w, sh, C═C), 1752 (m, sh, C═O), 1730 (s, sh, C═O), 1692 (s, sh, N-H). UV-Vis /nm (CDC1₃); λₘₐₓ = 280.

1H-Imidazole-1-carbohydrazide

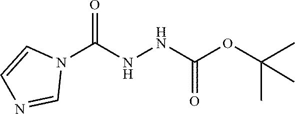 [SMILES: CC(C)(C)OC(=O)NNC(=O)n1ccnc1]

tert-butyl carbazate (595 mg, 4.5 mmol) was dissolved in water (15 ml) at room temperature and cooled to 0-5° C. Then, carbonyldiimidazole (1459 mg, 9.0 mmol) was added and the reaction mixture stirred for 1 hr at 0-5° C. and then allowed to warm up to room temperature. The precipitated product was then filtered and washed with cold water 3 to 4 times and dried to obtain a white powder (871 mg, 85% yield). ¹H NMR (400 MHz, DMSO-d6) / ppm: δ 10.43 (s, 1H, H-N), 9.27 (s, 1H, H-N), 8.27 (d, J= 1.2 Hz, 1H, H-C), 7.69 (t, J= 1.5 Hz, 1H, H-C), 7.03 (m, 1H, H-C), 1.44 (s, 9H, H-C). ¹³C NMR (101 MHz, DMSO-d6) / ppm: δ 155.84 (OC(O)N), 136.52 (NCH═HCN), 130.48(NCH═HCN), 116.95 (NCH═N), 80.43 (OCC2), 28.46 (CCH3).

N-propargyl-N′-(tert-butoxycarbonyl)amino urea PBocAU

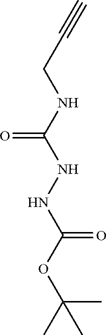

1H-Imidazole-1-carbohydrazide (566 mg, 2.5 mmol) and propargyl amine (248 mg, 4.5 mmol) were dissolved separately in EtoAc (15 mL) at room temperature. The two solutions were combined, and the mixture was stirred at room temperature overnight. The reaction was quenched with 0.5 M HC1 (20 mL). The organic layer was separated and washed with 0.5 M HC1 (2 × 20 mL) with deionized H₂O (6 × 20 mL) and brine (1 × 20 mL), and dried over sodium sulphate. The solvent was then removed by rotary evaporated to give an off-white colour powder at (241 mg, 45% yield). ¹H NMR (400 MHz, DMSO-d6) / ppm: 8 8.53 (s, 1H, H-N), 7.77 (s, 1H, H-N), 6.65 (s, 1H, H-N), 3.78 (dd, J= 5.8, 2.5 Hz, 2H, H2—CC═), 3.03 (t, J= 2.5 Hz, 1H, H—C≡), 1.40 (s, 9H, H-C). ¹³C NMR (101 MHz, DMSO-d6) / ppm: δ 156.38 (NC(O)N), 82.79 (CC═C), 79.45 (CCO), 72.86 (C═CH), 29.17 (HNCH2C═), 28.55 (CCH3).

1-(Chloromethyl)-4-ethynylbenzene

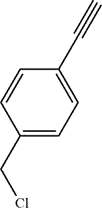

1-(hydroxymethyl)-4-ethynylbenzene (3.00 g, 22.7 mmol) dissolved in CHC1₃ (105 mL) and cooled to 0° C., methanesulfonyl chloride (1.2 mol equiv.) and Et3N (1.2 mol equiv.) were dissolved in the reaction mixture, and then heated to reflux for 45 h. Reaction was quenched with sat. NaHCO3, the organic layer was further washed with sat. NaHCO₃, dried with Na₂SO₄, filtered and concentrated in vacuo. The crude was purified by column chromatography (20:1 hexane/ EtOAc) to afford a pale yellow oil as the product (2.50 g, 73% yield). 1H-NMR (400 MHz, CDC13) 8(ppm): 7.50 (d, J= 8 Hz, 2H), 7.36 (d, J= 8 Hz, 2H), 4.58 (s, 2H), 3.12 (s, 1H).

N-(ethynylbenzyl)oxy)phthalimide EBPHT

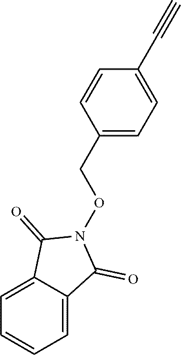 [SMILES: C#Cc1ccc(CON2C(=O)c3ccccc3C2=O)cc1]

N-Hydroxyphthalimide (2.17 g, 13.3 mmol) and Na₂CO₃ (1 mol equiv.) were dissolved in a mixture DMF, MeCN and water (43.5 mL, 0.457: 0.1875: 0.457 ratio). 1-(Chloromethyl)-4-ethynylbenzene (1 mol equiv.) was added to reaction mixture forming a deep red suspension and reacted for 4 h at room temperature. The mixture was filtered off and the precipitate was washed with water (100 mL × 3) and ice-cold methanol (100 mL × 3) to afford a white powdered solid as the product (3.58 g, 97% yield). ¹H-NMR (400 MHz, DMSO-d6) 8(ppm): 7.86 (s, 4H), 7.57 - 7.46 (m, 4H), 5.19 (s, 2H), 4.25 (s, 1H)

### Oligoethylene Glycol Containing Monomers

1-(Bromomethyl)-4-ethynylbenzene

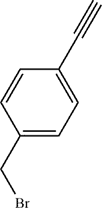

1-(Hydroxymethyl)-4-ethynylbenzene (4.85 g, 37.83 mmol) dissolved in THF (162 mL) and cooled to 0° C. in an ice bath for 20 minutes. Phosphorous tribromide (0.5 equiv.) was added dropwise to the solution and allowed to stir for an additional 15 minutes and then brought gradually to room temperature. The reaction was stirred at room temperature for 6 h, after which the solvent was removed in vacuo and the crude oil was purified by column chromatography (10 % EtOAc/ hexane) to afford an orange oil as the product (5.61 g, 76% yield). ¹H-NMR (400 MHz, CDC13) 8(ppm): 7.49 (d, J= 8.4, 2H), 7.37 (d, J = 8.4, 2H), 4.50 (s, 2H), 3.15 - 3.11 (s, 1H).

a-Ethynylbenzyl) tri(ethylene glycol)

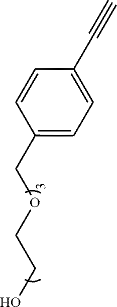

A solution of tri(ethylene glycol) (6.65 g, 43.85 mmol) in anhydrous THF (10 mL) was added to a suspension of NaH (438.4 mg, 10.96 mmol) in THF (40 mL)and heated to reflux. Then, 1-(bromomethyl)-4-ethynylbenzene (2.14 g, 10.96 mmol) was added dropwise to this suspension and refluxed for a further 3 h. The reaction was then cooled to room temperature, quenched with methanol (10 mL) and 5% HC1 (50 mL) and the solvent removed under vacuum. The product was then extracted with CHC1₃ (3 × 25 mL) and 5% HC1 (50 mL), dried with Na₂SO₄, filtered and concentrated in vacuo. The crude was purified by column chromatography (9:1 hexane/ EtOAc) to afford a colourless oil as the product (2.09 g, 72% yield). ¹H-NMR (400 MHz, DMSO-d6) 8(ppm): 7.49 - 7.40 (d, J = 8 Hz, 2H), 7.40 - 7.30 (d, J= 8 Hz, 2H), 4.57 (t, J= 5.5 Hz, 1H, OH), 4.52 (s, 2H), 4.17 (s, 1H), 3.60 - 3.39 (m, 12H).

a-Ethynylbenzyl) di(ethylene glycol)

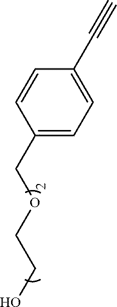

The same synthetic procedure and ratios were used as those described for a-(4-ethynylbenzyl) tri(ethylene glycol), but di(ethylene glycol) was used instead of tri(ethylene glycol). The crude was purified by column chromatography (9:1 hexane/ EtOAc) to afford a colourless oil as the product (1.90, 84% yield). ¹H-NMR (300 MHz, DMSO-d6) 8(ppm): 7.46 (d, J= 8.3 Hz, 2H), 7.35 (d, J= 8.49 Hz, 2H), 4.59 (t, J= 4.5 Hz, 1H, OH), 4.51 (s, 4H), 3.57 (s, 4H), 3.15-3.13 (m, 4H).

a-Ethynylbenzyl ethylene glycol)

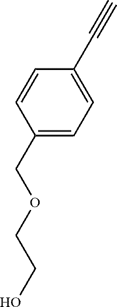

The same synthetic procedure and ratios were used as those described for a-(4-ethynylbenzyl) tri(ethylene glycol), but ethylene glycol was used instead of tri(ethylene glycol). The crude was purified by column chromatography (9:1 hexane/ EtOAc) to afford a colourless oil as the product (84% yield). ¹H-NMR (300 MHz, CDC13) 8(ppm): 7.48 (d, J= 7.99, 2H), 7.30 (d, J= 8.55, 2H), 4.56 (s, 2H), 3.87 - 3.70 (m, 2H), 3.66 - 3.55 (m, 2H), 3.07 (s, 1H), 2.01 (m, 1H, OH).

a-Ethynylbenzyl) co-tosyl tri(ethylene glycol)

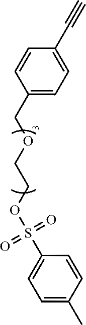

a-(4-Ethynylbenzyl) tri(ethylene glycol) (1 g, 3.78 mmol) and NaOH (3.5 mol equiv.) were dissolved in a dry THF and distilled water mixture (5.5 mL, 1:1 ratio) and cooled to 0° C. A separate THF solution (3.5 mL) ofp-toluenesulfonyl chloride (874.3 mg, 4.54 mmol) was then added dropwise to the stirred suspension over the course of 30 mins and was stirred at 0° C. for a further 2 h, before allowing it to warm to room temperature and reacted for a further 20 h. The reaction was then cooled to 0° C., poured to 5% HC1 (50 mL) on ice and extracted with CHC1₃ (3 × 25 mL), dried with Na₂SO₄, filtered and concentrated in vacuo to afford a colourless oil as the product. This product was then used without further purification in subsequent syntheses (1.84 g, 100% yield; 90% purity). ¹H- NMR (300 MHz, CDC13) 8(ppm): 7.85 - 7.72 (m, 2H), 7.49 - 7.43 (m, 2H), 7.36 - 7.27 (m, 3H), 4.55 (s, 2H), 4.18 - 4.11 (m, 2H), 3.74 - 3.60 (m, 3H), 3.06 (s, 1H), 2.43 (s, 3H).

a-Ethynylbenzyl) co-tosyl di(ethylene glycol)

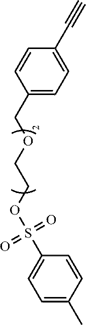

The same synthetic procedure and ratios were used as those described for a-(4-ethynylbenzyl) ω-tosyl tri(ethylene glycol)to give a colourless oil (1.68 g, 99% yield; 94% purity). ¹H-NMR (300 MHz, CDC13) 8(ppm): δ 7.84 - 7.74 (m, 2H), 7.51 - 7.41 (m, 2H), 7.35 - 7.28 (m, 4H), 4.53 (s, 2H), 4.21- 4.13 (m, 2H), 3.80 - 3.52 (m, 6H), 3.07 (s, 1H), 2.43 (s, 3H).

a-Ethynylbenzyl) co-phthalimide tri(ethylene glycol) EBTEGPHT

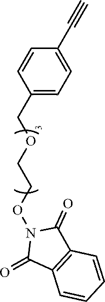

a-(4-Ethynylbenzyl) ω-tosyl tri(ethylene glycol) (1.0 g, 2.39 mmol) and N-hydroxyphthalimide (1.2 mol equiv.) were suspended in dry MeCN (9 mL). Et3N (1.2 mol equiv.) was added to afford a deep red solution. The reaction was heated to reflux and left for 17 h. Solvent was then removed under vacuum and the resultant crude oil was redissolved in CHC1₃ (50 mL) and washed with sat. NaHCO₃ (4 × 25 mL). The organic phase was dried with Na₂SO₄, filtered and concentrated in vacuo to afford a crude yellow oil. The product was then purified by flash chromatography (1:1 hexane/EtOAc) to afford a yellow oil as the product (730.6 mg, 75% yield). ¹H NMR (400 MHz, DMSO-d6) 8(ppm): 7.86 (s, 4H), 7.49 - 7.40 (m, 2H), 7.39 -7.28 (m, 2H), 4.49 (s, 2H), 4.32 - 4.23 (m, 2H), 4.16 (s, 1H), 3.78 - 3.68 (m, 2H), 3.60 - 3.41 (m, 8H).

a-Ethynylbenzyl) co-phthalimide di(ethylene glycol) EBDEGPHT

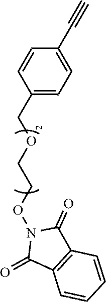

The same synthetic procedure and ratios were used as those described for a-(4-Ethynylbenzyl) ω-phthalimide tri(ethylene glycol). The product purified by flash chromatography (1:1 hexane/EtOAc) to afford a yellow oil as the product (1.05 g, 63% yield). ¹H-NMR (300 MHz, 20 CDC13) 8(ppm): 7.85 - 7.77 (m, 2H), 7.77 - 7.69 (m, 2H), 7.47 - 7.41 (m, 2H), 7.25 - 7.21 (m, 2H), 4.48 (s, 2H), 4.44 - 4.35 (m, 2H), 3.93 - 3.85 (m, 2H), 3.73 - 3.64 (m, 2H), 3.61 - 3.50 (m, 2H), 3.06 (s, 1H).

### Polymerisation of Acetylene Monomers

All polymerisation were performed in a similar manner to Masuda et al. (Polymerization of Substituted Acetylenes, in Handbook of Metathesis: Catalyst Development. (2008)). One of the four following rhodium catalysts were employed, [Rh(NBD)nPh—BPh₃], [(Rh(NBD)C1)₂], [(Rh(COD)C1)₂], and [Rh(COD)₂]^{⊕} BF₆^{⊖}. [Rh(nbd)B(Ph)₄] was synthesised as in Schrock, R. R.; Osborn, J. J. Inorg. Chem. 1970, 9, 2339.

In a typical procedure, protected monomer (PBocAC, PBocAU, EBPHT, EBTEGPHT, EBDEGPHT) (1.43 mmol) was dissolved in a suitable solvent (6 mL) at 30° C. When fully dissolved rhodium catalyst (0.0143 mmol, 0.01 mol equiv. per monomer, target x = 100) was added, either in solution or solid. The mixture was stirred at 30° C. for 24 hours. The reaction mixture was precipitated over excess n-hexane. The resulting precipitate was then dried on a rotary evaporated to produce a pale-yellow powder. Other experimental conditions for the synthesis of poly(acetylene)s are described in Masuda, T. et al. Polymerization of Substituted Acetylenes, in Handbook of Metathesis: Catalyst Development. (2008).

poly(O-propargyl-N-(tert-butoxycarbonyl)amino carbamate) p(PBocAC)

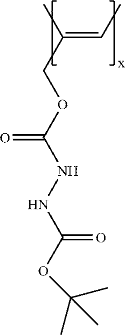

Following the polymerization conditions described above, PBocAC (4.67 mmol) and [Rh(nbd)B(Ph)₄] (0.01 mol equiv. per monomer0.01 mol equiv. per monomer) were reacted in THF (15.7 ml) to give 610 mg (61% yield) of the title compound. ¹H NMR (400 MHz, DMSO-d6) /ppm; 8.87 (s, 1H, H-H), 8.61 (s, 1H, H-N), 6.35 (s, 1H, H—C═), 4.66 (s, 2H, H-CC2), 1.38 (s, 9H, H-CC). ¹³C NMR (400 MHz, DMSO-d6) /ppm; 155.77, 155.63 & 155.52 (OC(O)N), 79.40, 77.56 (C═CH), 52.09 (C2CO), 28.05 (CCH3). FTIR vmax /cm⁻¹; 3290 (m, H-N), 2979 (w, sh, H-C), 1707 (s, sh, C=O). UV-Vis /nm (DMSO); λₘₐₓ = 288 nm. GPC; MwGPC = 28340 (±10673); MnGPC = 20115 (±7324); ÐGPC = 1.41 (±0.095).

poly(N-propargy1-N′-(tert-butoxycarbony1)amino urea) p(PBocAU)

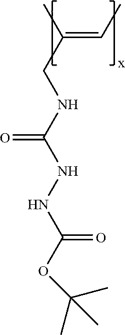

Following the polymerization conditions described above, PBocAU (1.43 mmol) was dissolved in chloroform (6 mL) at 30° C. When fully dissolved [Rh(nbd)B(Ph)₄] (0.01 mol equiv. per monomer) was added. 249 mg (96% yield) of the title compound were isolated. ¹H NMR (400 MHz, DMSO-d6) / ppm: δ 8.46 (s, 1H, H-N), 7.69 (s, 1H, H-N), 6.55 (s, 1H, H—N) 60.11 (s, 1H, H—C═), 3.78 (s, 2H, H—CCNH) 10.39 (d, J = 5.9 Hz, 9H).

polyr[N-((4-ethynylbenzyl)oxy)phthalimide] p(EBPHT)

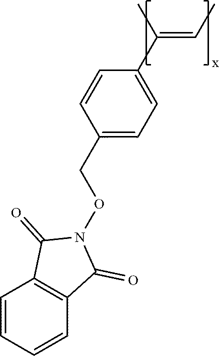

Following the polymerization conditions described above, EBPHT (1.07 mmol) was dissolved in DMF (6 mL) and Et3N (0.01 mol equiv.) at 30° C. When fully dissolved [Rh(nbd)B(Ph)₄] (0.01 mol equiv. per monomer) was added. The title polymer was precipitated into methanol, filtered, diluted in water then dialysed for four days. ¹H NMR (400 MHz, DMSO-d6) 8(ppm): 7.53 (br m, 4H), 7.29 - 6.88 (br s, 2H), 6.62 (br s, 2H), 5.70 (s, 1H), 5.27 - 4.41 (m, 2H).

polyra-(α-(4-Ethynylbenzy1) co-phthalimide di(ethylene glycol)] p(EBDEGPHT)

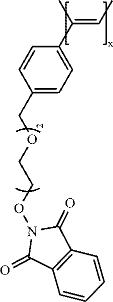

Following the polymerization conditions described above, EBDEGPHT (1.07 mmol) was dissolved in DMF (6 mL) and Et3N (0.01 mol equiv.) at 30° C. When fully dissolved [Rh(nbd)B(Ph)₄] (0.01 mol equiv. per monomer) was added. The title polymer was isolated by diluting in water, dialysed for four days, and freeze-drying. ¹H NMR (400 MHz, CDC13) δ(ppm): 8 7.59 - 7.48 (m, 4H), 6.72 (s, 2H), 6.44 (s, 2H), 5.63 (s, 1H), 4.60 (s, 1H), 4.21 - 4.05 (m, 2H), 3.63 (s, 2H), 3.39 (s, 2H), 3.21 (s, 2H).

### Deprotection of Acetylene-Based Polymers

Reactive polymers may then be produced by deprotection. Certain protecting can be removed under acidic conditions, e.g. the Boc groups of p(PBocAC) and p(PBocAU) to make p(PAC) and p(PAU) respectively. Other protecting groups must be deprotected using basic conditions, e.g. the phthalic acid protecting groups of p(EBPHT) and p(EBDEGPHT) to make (p(EBHA) and p(EBOEGHA). Other experimental conditions for the deprotection of amines, for example as carbamates or phthalimides, can be found in Peter G. M. Wuts Theodora W. Greene, Protection for the Amino Group in Protective Groups in Organic Synthesis (2007).

poly(O-propargyl-N-amino carbamate) p(PAC)

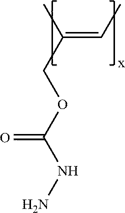

p(PBocAC) (1 g, 60.7 mmol) was dissolved in 10 ml TFA. The mixture was stirred for 2 hours at room temperature. Excess TFA was blown off with a steady stream of Argon. 10 ml of deionised water was added to the reaction vessel, and the solution was neutralised with sodium bicarbonate. The polymer was then dialysed against 100 mM acetic acid and freeze dried to yield a pale-yellow powder (465.3 mg, 46.53% yield) that was stored under vacuum. FTIR vmax /cm⁻¹; 3292 (m, br, H-N), 2950 (w, br, H-C), 1690 (s, br, C═O), 1616 (w, br, C═O). UV/Vis /nm (HC1); λₘₐₓ = 275. Fluorescence /nm (HC1); λexc = 355, 385, 413; λem = 491. LD (Thin film) /dOD @ nm; 4.9 ×10⁻⁴ @ 510. DSC / °C; 139, 205.

poly(N-propargyl-N′-amino urea) p(PAU)

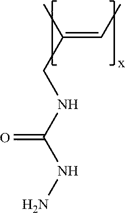

p(PBocAU) (249 mg, 2.6 mmol) was dissolved in a minimum amount of TFA, and the mixture stirred for 20 hr at room temperature. The TFA was then removed by a steady stream of Argon until the high viscosity of the reaction mixture is seen. Thereafter, the solution was neutralised with sodium bicarbonate. The polymer was then dialysed again 100 mmol acetic for 4-5 days and freeze-dried to give a pale-yellow powder (149 mg, 60% yield). ¹H NMR (400 MHz, DMSO-d6) / ppm: 8 9.68 (s, 2H, H2-N), 9.13 (s, 1H, H-N), 7.42 (s, 1H, H-N), 6.14 (s, 1H, H—C═), 3.88 (s, 1H, H-CCNH).

### Synthesis of Polymers of Formula (I)

To a solution of reactive polymer (e.g. pAH, p(PAC), p(PAU), p(EBHA), p(EBOEGHA)) in solvent (200 µl, 100 mM reactive polymer solution in reactive functional group (e.g. hydrazide) moieties as determined by ¹H NMR; by default, for example, for pAHx, x may be 50) was added an aldehyde in a water-miscible solvent (200 µl, 100 mM). This mixture was shaken at 60° C. for 3 to 24 h. Polymers were used without further purification.

Details of specific examples are shown in the table below

| Designation of Polymer | Aldehyde | Solvent | % functionalisation | Polymer structure |
|---|---|---|---|---|
| pAH-2AFP | 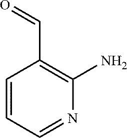 | 100 mM AcOH in D₂O (pH 2.9) | 61 | 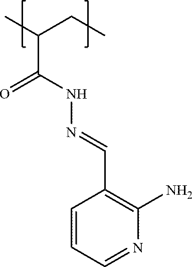 |
| pAH-IMI | 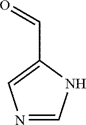 | 100 mM AcOH in D₂O (pH 2.9) | 75 | 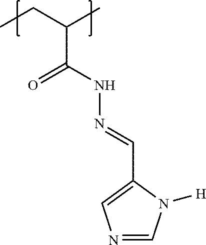 |
| pAH-IVA | 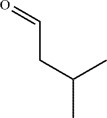 | 95 vol.% DMSO-d₆ 5 vol.% 100 mM AcOH | 80 | 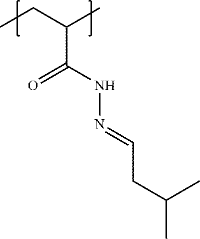 |
| pAH-Bn | 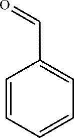 | 95 vol.% DMSO-d₆ 5 vol.% 100 mM AcOH | 65 | 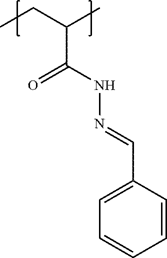 |
| pAH-In | 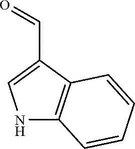 [SMILES: O=Cc1c[nH]c2ccccc12] | 95 vol.% DMSO-d₆ 5 vol.% 100 mM AcOH | 53 | 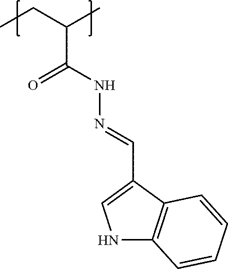 |
| pAH - Pyr | 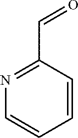 | 95 vol.% DMSO-d₆ 5 vol.% 100 mM AcOH | 63 | 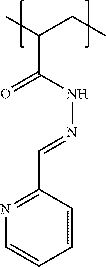 |
| pAH - Naph | 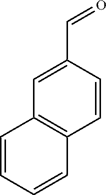 [SMILES: O=Cc1ccc2ccccc2c1] | 95 vol.% DMSO-d₆ 5 vol.% 100 mM AcOH | 62 | 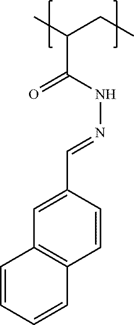 |
| pAH - Anthr | 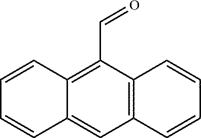 [SMILES: O=Cc1c2ccccc2cc2ccccc12] | 95 vol.% DMSO-d₆ 5 vol.% 100 mM AcOH | 54 | 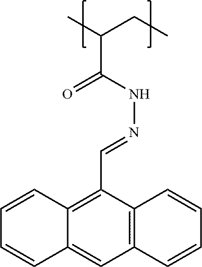 |
| pAH - Pyrene | 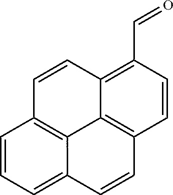 [SMILES: O=Cc1ccc2ccc3cccc4ccc1c2c34] | 95 vol.% DMSO-d₆ 5 vol.% 100 mM AcOH | 58% | 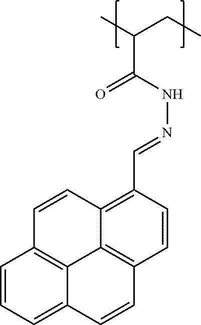 |
| p(PAC)-2AFP | 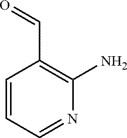 | 50 vol.% DMSO-d₆ 50 vol.% 0.1 M HC1, pH 3 | 74 | 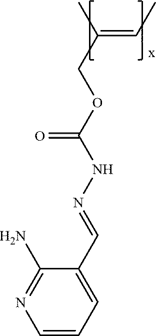 |
| p(PAC)-IMI | 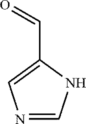 | 50 vol.% DMSO-d₆ 50 vol.% 0.1 M HC1, pH 3 | 72 | 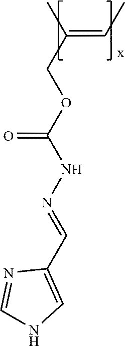 |
| p(PAC)-IVA | 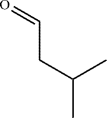 | 50 vol.% DMSO-d₆ 50 vol.% 0.1 M HC1, pH 3 | 78 | 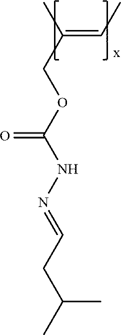 |
| p(PAC)-Bn | 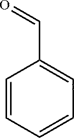 | 50 vol.% DMSO-d₆ 50 vol.% 0.1 M HC1, pH 3 | 61 | 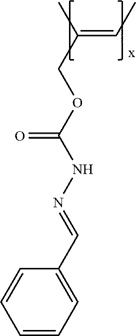 |
| p(PAC)-In | 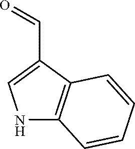 [SMILES: O=Cc1c[nH]c2ccccc12] | 50 vol.% DMSO-d₆ 50 vol.% 0.1 M HC1, pH 3 | 64 | 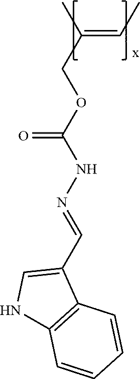 |

It will be appreciated that similar yields of imine-containing compounds may be achieved using the variety template polymers described above, which have for example different backbones and different linker groups. Therefore, a wide variety of polymers of formula (I) can be synthesised using the disclosure of the present application.

It has been recognised that when the loading of aldehyde is increased, in this example specifically from 1 equivalent to 2 equivalents of aldehyde, the % functionalisation of the resulting polymer increases.

### General Procedure for Production of Microorganism-Polymer Complex

The microorganism (e.g. Escherichia ( E.) coli) was grown overnight in LB broth at 30 degrees and stirred with an orbital shaker with a throw of 19 mm at 150 rpm. The resulting culture was re-inoculated with fresh LB (1% re-inoculation) and grown in the same conditions for around 3 h until the culture reached an OD of 0.2. The cells were washed in water and isolated by centrifuge. The isolated cells were suspended in sufficient KH₂PO₄/K₂HPO₄ buffer (0.1 M, pH 7.0) to give an OD of 0.2. The polymer was added to the resulting solution. Then M63 media (equal volume to that of the KH₂PO₄/K₂HPO₄) was added. The final polymer concentration was determined to be 0.053 mg/mL. The culture was incubated at 30 degrees and mixed with an orbital shaker with a throw of 19 mm at 150 rpm for 24 h or 48 h.

### Investigations Relating to Microorganism-Polymer Complexes

### Size Distribution of Complexes

PHL644 E. coli were grown overnight in LB media. The next morning, the cells were washed with water and re-suspended in KH₂PO₄/K₂HPO₄ (0.1 M in deionised water) to an OD of 1.0. Half of the cells were then mixed with polymer pAH-2AFP (final concentration 0.5 mg/ml). The two samples of cells (i.e. with and without pAH-2AFP) were mixed for 48 h before the size distribution was determined.

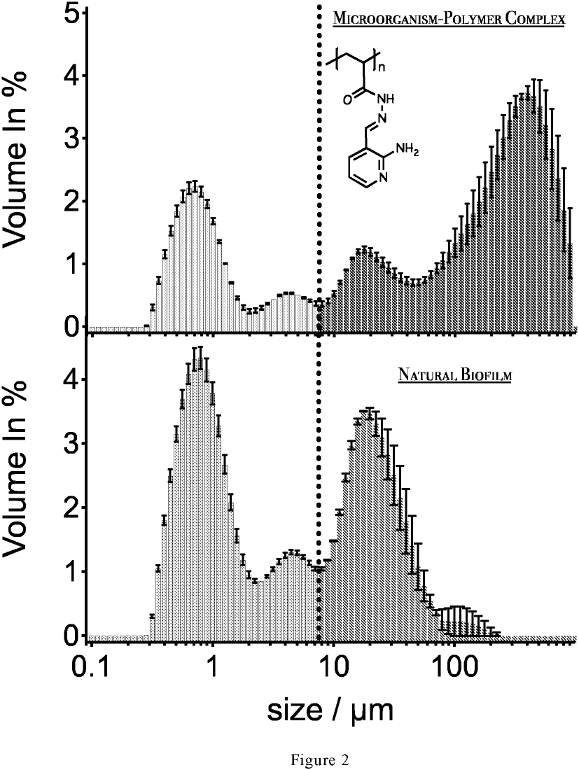

FIG. 2 shows that after 48 hours the sample of E. coli that was not mixed with the polymer (bottom graph) retained a large proportion of planktonic cells. There were some small clusters of natural biofilm, having a peak size of about 20 µm. There were few clusters greater in size than 200 µm, but no recorded clusters greater in size than 300 µm.

The E. coli that was mixed with pAH-2AFP (top graph) had a lower proportion of planktonic cells and had a higher proportion of clusters of high diameter, up to 1000 µm in size.

This indicates that the polymer formed complexes with the cells of the microorganism.

### Crystal Violet Staining

The interaction of polymers according to the invention and E. coli was also studied by Crystal Violet staining. Crystal Violet (CV) is employed to stain the extracellular material in biofilms (in particular the negatively charged components of bacterial cells envelopes and extracellular matrices). This technique allows the amount of biomass in a culture to be correlated with microorganism-polymer complex formation.

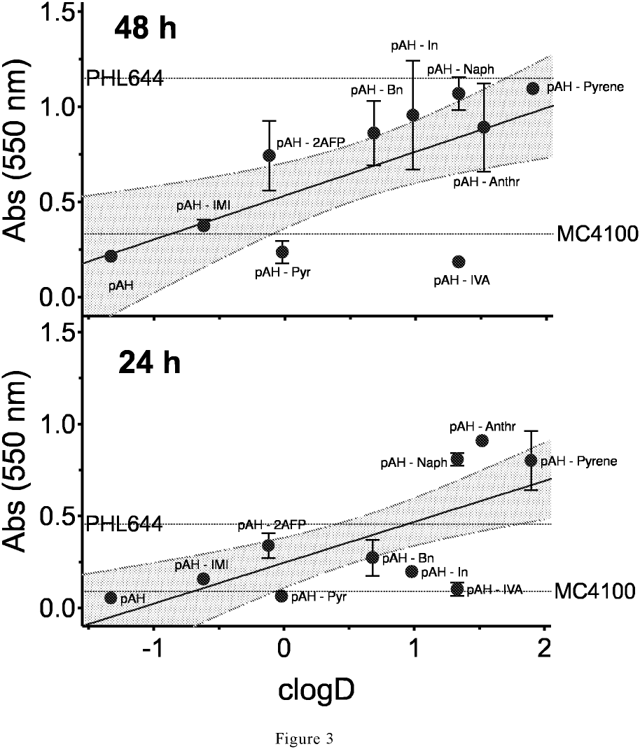

Cultures of weak biofilm-forming E. coli strain MC4100 with pAH or polymers of the invention were washed with deionised water and the supernatant was discarded. The washed residue was stained with 1% crystal violet solution for 1 h. The stained residue was washed three times with deionised water, discarding the supernatant from each wash, to remove excess crystal violet. The stained and washed residue was dissolved in a controlled volume of 33% acetic acid and the absorbance at 550 nm was measured using a ClarioStar Plus BMG plate reader. In a control experiment, the bacteria was not mixed with any polymer but was exposed to Crystal violet. The amount of complex formation by MC4100 E. coli, in the presence of pAH or polymers of the invention was determined using CV staining at 24 and 48 hours. These results were correlated with the clogD value calculated for the R² group of the polymer. FIG. 3 shows the results of this analysis.

It was observed that each of the cultures formed complexes akin to biofilms in the presence of the polymers of the invention.

Therefore, it is possible to produce biofilm-like microorganism-polymer complexes in cultures with the polymers of the present invention.

Furthermore, cultures including certain polymers exhibited significantly more cluster formation than the control culture at 24 hours and/or 48 hours (MC4100 24 h, MC4100 48 h). These polymers were pAH-AFP, pAH-IMI, pAH-Bn, pAH-In, pAH-Naph, pAH-Anthr and pAH-Pyrene.

Surprisingly, where a polymer selected from pAH- AFP, pAH-Bn, pAH-In, pAH-Naph, pAH-Anthr and pAH-Pyrene was used, the cultures of MC4100 reached levels of absorbance approaching, equalling or even improving upon control cultures of the stronger biofilm-forming strain PHL644 at 24 hours and/or 48 hours.

Surprisingly, where a polymer selected from pAH-Naph, pAH-Anthr and pAH-Pyrene was used, the cultures of MC4100 reached levels of absorbance significantly improving upon control cultures of the stronger biofilm-forming strain PHL644 at 24 hours.

Crystal violet staining of the complex may not be the only metric to determine the most useful polymers, as the polymers producing lower crystal violet staining may produce stronger complexes with microorganisms and/or be more beneficial for other strains and/or species of microorganism.

Thus, as shown by FIG. 3, it is possible to produce biofilm-like clusters, microorganism-polymer complexes, in cultures with the polymers of the present invention.

### Curli Expression

Curli is a type of amyloid fibre produced by certain strains of enterobacteria. They are extracellular fibres located on bacteria such as E.coli and Salmonella. Curli are primarily known to be the adhesin by which cells attach to biotic and abiotic surfaces. There is evidence that Curli proteins are involved in cell-cell attachment. Thus, Curli expression may be indicative of the formation of a microorganism-polymer complex.

MC4100 E. coli was genetically modified to express GFP (green fluorescent protein) as a function of Curli expression. A plasmid containing a gene reporter for the csgBAC operon (pJLC-A) was used, as described by James Thomas Leech “Development of an Escherichia coli Biofilm Platform for use in Biocatalysis”, PhD thesis, University of Birmingham (UK), 2017, https://etheses.bham.ac.uk/id/eprint/8055/1/Leech18PhD.pdf accessed 11 Oct. 2019 (especially pages 70-76).

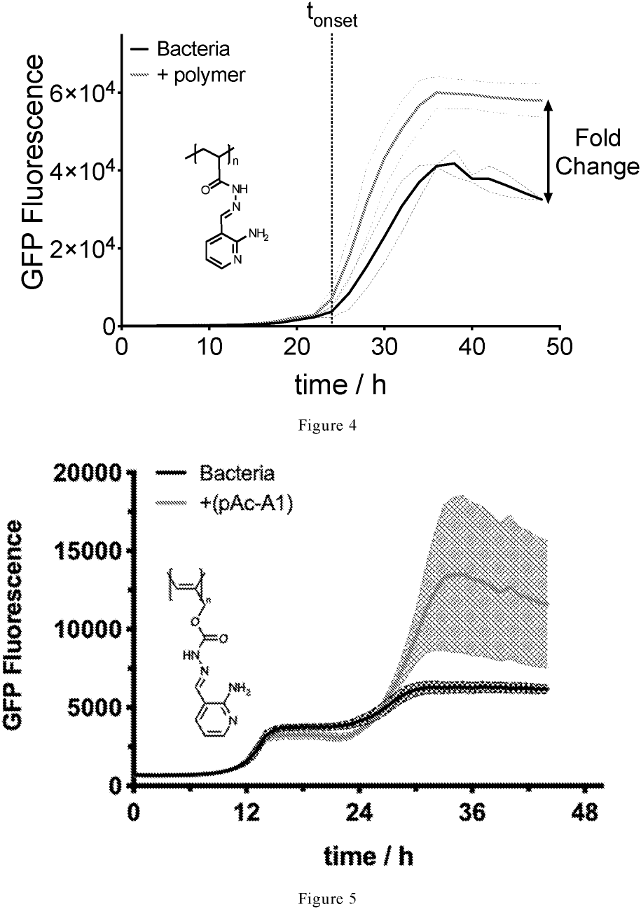

The fluorescence of the genetically modified E. coli was measured over 48 hours in the presence and absence of polymer pAH-2AFP using a BMG Clariostar plus (excitation wavelength 488 nm; emission wavelength 510 nm). This allowed for measurement of Curli expression, and therefore monitoring of the microorganism-polymer complex formation by E. coli in the presence or absence of polymer. The results of this analysis are shown in FIG. 4.

The results show that for an initial period of about 24 hours the fluorescence of the E. coli was low in the bacteria with or without pAH-2AFP. Without being bound by theory, this may be because the MC4100 strain of E. coli used in this experiment naturally produces Curli slowly, or because it takes time for the cells to respond to the environmental stimuli that initiate biofilm formation.

After about 24 hours the fluorescence of both cultures began to increase until it reached a plateau at about 35 to 40 hours. The culture including the polymer reached a maximum fluorescence of about 150% of the maximum fluorescence of the culture without the polymer.

Therefore, culturing microorganisms with a polymer as defined by formula (I) can produce higher levels of biofilm-like microorganism-polymer complex, than without the polymer.

The fluorescence of the culture without the polymer decreased up to the 48 hour point. However, whilst some decrease in the fluorescence of the culture with the polymer was observed, this decrease was less severe than in the sample without the polymer.

This suggests that the complex in the culture with the polymer is more stable than the biofilm in the sample without the polymer.

The same experiment was performed with the carbazate hydrazone version of pAH-2AFP (shown below) according to a similar procedure to that reported above.

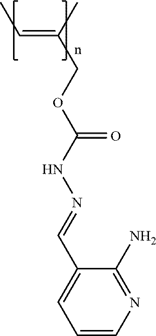

FIG. 5 shows the fluorescence by GFP over time.

The results show that for an initial period of about 24 hours the fluorescence of the E. coli was similar for the bacteria with and without the polymer. After about 24 hours the fluorescence of both cultures began to increase further until it reached a plateau at about 30 to 36 hours. The culture including the polymer reached a maximum fluorescence of about double the maximum fluorescence of the culture without the polymer, indicating significantly more formation of biofilm-like structures in the presence of the polymer.

Therefore, culturing microorganisms with a polymer as defined by formula (I) that contain a carbazate hydrazone group as the linker can produce higher levels of biofilm-like microorganism-polymer complex than without the polymer.

The normalised GFP expression of cultures of MC4100 and PHL644 strains of E. coli was determined after 48 hours with or without pAH-2AFP.

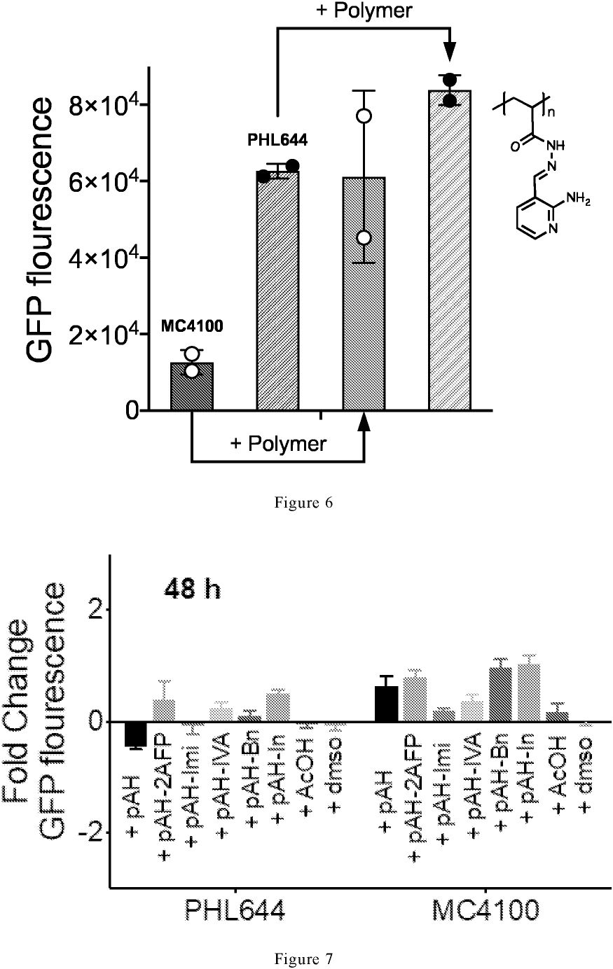

As shown by FIG. 6, each of these E. coli strains had significantly higher normalised GFP expression when pAH-2AFP was present. MC4100, a poor biofilm forming strain of E. coli, was able to produce a strong microorganism-polymer complex with pAH-2AFP. PHL644, already a strong biofilm forming strain of E. coli, was able to produce an even stronger microorganism-polymer complex with pAH-2AFP.

This shows that the polymers of the present invention enable weak biofilm-forming microorganisms to be used in applications that have been developed for, or are more suited towards, (stronger) biofilms/microorganism-polymer complexes.

It also shows that strong biofilm forming microorganisms can produce stronger biofilm-like complexes when they are cultured with a polymer, according to the present invention.

The GFP fluorescence of cultures with polymer pAH or a polymer of the invention over 48 hours was determined with PHL644 and MC4100 strains of E. coli. The fold change in fluorescence of the culture with each polymer was determined and is shown in FIG. 7. As indicated by FIG. 7, the “fold change” of a polymer refers to the GFP fluorescence of that polymer in the culture at 48 hours divided by that of the control sample. The unmarked bars signify control experiments with either DMSO or AcOH.

The results show that, in most cases, the polymers of the invention significantly increased the fluorescence of the culture. Every polymer of the invention increased the fluorescence of the MC4100 culture. Thus, it is currently proposed that biofilm-like characteristics of a microorganism are enhanced by polymers of the present invention.

It can be seen that polymers pAH-2AFP and pAH-In provided the best results with E. coli strain PHL644. These polymers each include a heteroaromatic ring in the R² group. It can be seen that polymers pAH-2AFP, pAH-Bn and pAH-In provide the best results with E. coli strain MC4100.

The same experiment was performed with polymers that contained units of poly(acetylene) backbone and carbazate hydrazone linkers:

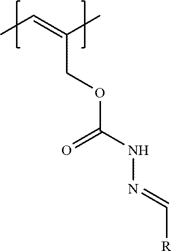

Where the R groups were:

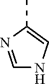

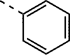

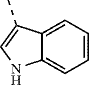

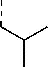

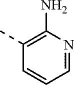

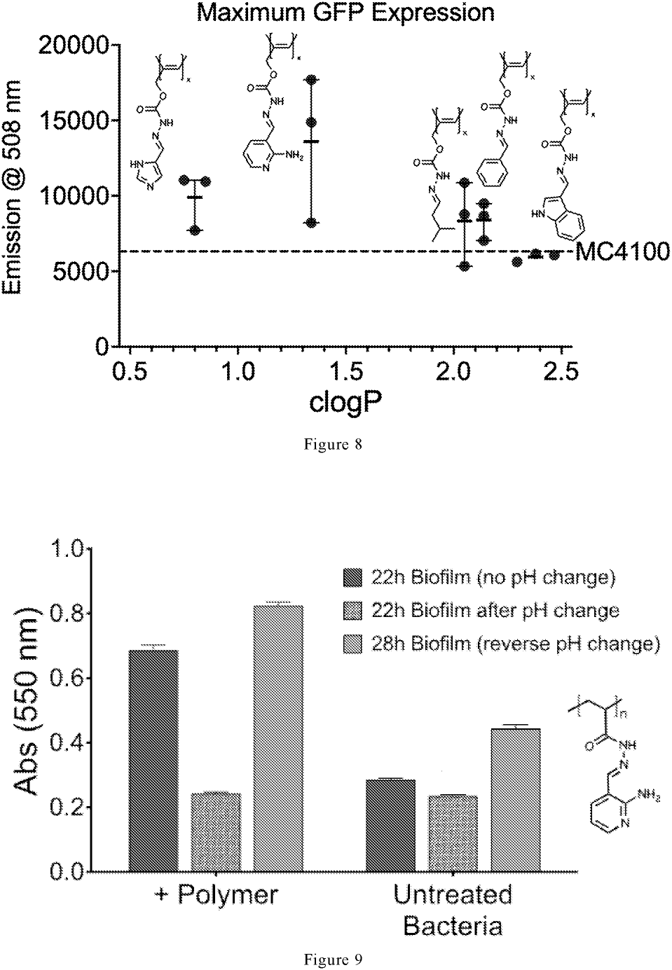

FIG. 8 shows the maximum GFP expression following incubation of these polymers with MC4100, as described above, correlated against the clogP for the monomer unit of the polymer. A dashed line shows the baseline emission for a comparative culture of MC4100.

It can be seen that there is generally a decrease in the expression as clogP increases, indicating that formation of the complex is lower when polymers with monomer units having a higher clogP were used. Therefore, it may be beneficial to use polymers with monomer groups having a low clogP to encourage matrix formation with these bacteria.

The polymers including, as R² groups, imidazolyl, 2-aminopyridyl, phenyl and s-butyl groups provided more matrix formation than the comparative culture.

### Reversibility of Microorganism-polymer Complex Formation with Polymers

Investigations were performed into the reversibility of microorganism-polymer complex formation using E. coli strain PHL644 with polymer pAH-2AFP.

A sample of the microorganism was exposed to pAH-2AFP under the normal conditions detailed above. A control sample of the microorganism was subjected to the same conditions but without pAH-2AFP. After incubation for 22 hours, the amount of biofilm/complex formed in the sample with pAH-2AFP and the control sample was determined by crystal violet staining and absorbance measurement at 550 nm.

The results of this analysis are shown by the bars labelled “22 h Biofilm (no pH change)” in FIG. 9. There was an increase in absorbance for the sample with pAH-2AFP compared to the control culture. This relative increase in absorbance suggests that the biofilm-like microorganism-polymer complex had formed.

Two fresh samples of the microorganism were exposed to pAH-2AFP under the normal conditions detailed above. After incubation for 16 hours the samples were acidified from the usual pH of about 7.0 to 5.8 by adding KH₂PO₄ (https://www.unl.edu/cahoonlab/phosphate%20buffer.pdf, accessed 11 Oct. 2019). The samples were incubated for a further 6 h. Two control samples of the microorganism were subjected to the same conditions but without pAH-2AFP. The amount of biofilm/complex formed in one sample with pAH-2AFP and one control sample was determined by crystal violet staining and absorbance measurement at 550 nm.

As shown by the bars labelled “22 h Biofilm after pH change” in FIG. 9, the absorbance of the sample with pAH-2AFP at this stage was similar to that of the control sample, suggesting that the microorganism-polymer complex had been at least partially, or fully, dispersed by the change in pH. This also suggests that the amount of bacteria in the microorganism-polymer complex form may be able to be decreased by controlling (for example decreasing) the pH of the culture, where the culture includes a polymer according to the present invention. Where the amount of bacteria in the microorganism-polymer complex form has an effect on, for example, the rate of a reaction that is mediated by the microorganism-polymer complex, the rate of reaction may be decreased by controlling (for example decreasing) the pH of the culture.

The acidified samples that were not stained, i.e. one control sample and one sample with pAH-2AFP, were treated with K₂HPO₄ to restore their pH to 7.0, and the resulting samples were incubated for another 6 h before and biofilm/complex formation was determined by crystal violet staining and absorbance measurement at 550 nm.

As shown by the “28 h Biofilm (reverse pH change)” bars in FIG. 9, an increase in absorbance was observed in each sample, suggesting an increase in the amount of microorganism-polymer complex/biofilm. However, it is notable that the absorbance of the culture containing pAH-2AFP increased by about three times, whereas the absorbance of the control culture did not increase by even two times.

This indicates that the culture containing pAH-2AFP was able to reform a microorganism-polymer complex, whereas the control culture remained in a predominantly planktonic form.

Thus, the amount of bacteria in the microorganism-polymer complex may be able to be controlled (i.e. decreased and/or increased) by adjusting (i.e. decreasing and/or increasing) the pH of the culture, where the culture includes a polymer according to the present invention. Where the amount of bacteria in the microorganism-polymer complex form has an effect on, for example, the rate of a reaction that is mediated by the microorganism-polymer complex, the rate of reaction may be controlled by the pH of the culture.

Without being bound by theory, it is postulated that the decrease in pH after the initial 22 hours of incubation hydrolysed the imine bonds of the polymer. Thus, it is postulated that the R² groups of the polymer were detached from the polymer backbone. This may mean that the R² groups would no longer hold the cells in a complex, and the complex was dispersed. The increase in absorbance after the pH was returned to its initial value suggests that the cells were held in a complex once more. This may be because the polymer backbone had reattached to the R² groups.

### Use of Complexes to Perform Microorganism-Mediated Reactions

### Transformation of 5-fluoroindole Into 5-fluorotryptophan

The influence of polymers according to the present disclosure on the microorganism-mediated transformation of 5-fluoroindole into 5-fluorotryptophan mediated was studied. This biotransformation has been described previously using a range of spin coated E. coli K-12 biofilms matured in M63 medium for 7 days. As an example, MC4100 spin coated biofilms were able to convert 10% of 5-fluoroindole to 5-fluorotryptophan after 24 h, whereas the higher biofilm forming PHL644 was able to convert 50% in 24 h (FIG. 5 of Perni et al., AMB Express, 2013, 3:66).

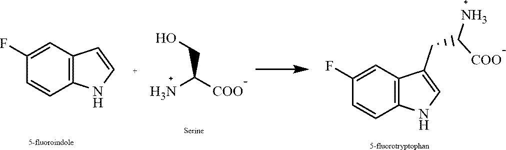

The microorganism-polymer complex was prepared for pAH and polymers according to the invention with E. coli strain MC4100, as described above, and left for 48 h to stabilise. The microorganism-polymer complexes were isolated by centrifuge and washed with KH₂PO₄/K₂HPO₄ buffer (0.1 M). The supernatants were removed and 1 ml of reaction buffer (0.1 M KH₂PO₄/K₂HPO₄, 7 mM serine, 1 mM PLP and 1.5 mM fluoroindole) was added to each microorganism-polymer complex. Each sample of complex in the reaction buffer was left to react for 24 h.

The conversion of fluoroindole to fluorotryptophan was determined according to the following equation:

$$\% C o n v e r s i o n = \frac{Y i e l d o f f l u o r o t r y p t o p h a n \left(\%\right)}{D e p l e t i o n o f f l u o r i i n d o l e \left(\%\right)} \times 100$$

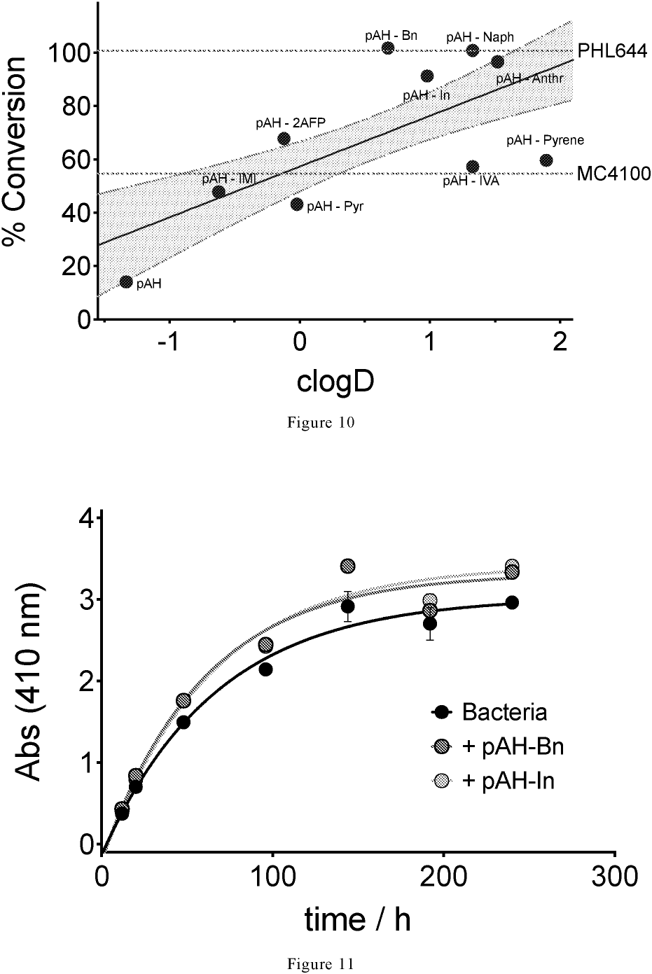

The conversion for the sample containing each polymer is shown in FIG. 10.

FIG. 10 shows that the microorganism-polymer complexes of the present invention can be used to perform chemical transformations.

It was surprising that cultures including complexes with polymers according to the invention each provided conversion exceeding that reported in the literature.

It was also surprising that cultures including complexes with polymers pAH-Bn, pAH-In, pAH-Naph or pAH-Anthr provided exceptional conversions, similar to that seen when using the strong biofilm-forming strain PHL644.

This shows that the performance of weaker biofilm-forming microorganisms can be improved by culturing with a polymer as defined by formula (I).

As shown by the trend line, as clogD of the R² group increases, the conversion generally increases. Thus, there may be a correlation between conversion and the clogD of the monomer unit.

### Hydrolysis of 4-nitrophenyl Dodecanoate

E. Coli Can Naturally Produce Esterase/lipase Enzymes, Which Hydrolyse Esters.

Samples of microorganism-polymer complexes were prepared as described above and left incubating at 30 degrees, 150 rpm for 48 h. 4-nitrophenyl dodecanoate (as a solution in isopropyl alcohol, to make a final concentration of 0.08 mM of 4-nitrophenyl dodecanoate and 50% isopropyl alcohol by volume) was added. Esterase activity was monitored by monitoring absorbance of the hydrolysis product, 4-nitrophenol, at 410 nm over 240 h.

The significant amount of isopropyl alcohol used in this experiment was considered to be challenging to the microorganism. This was used to dissolve the 4-nitrophenyl dodecanoate as this chemical is poorly soluble in water.

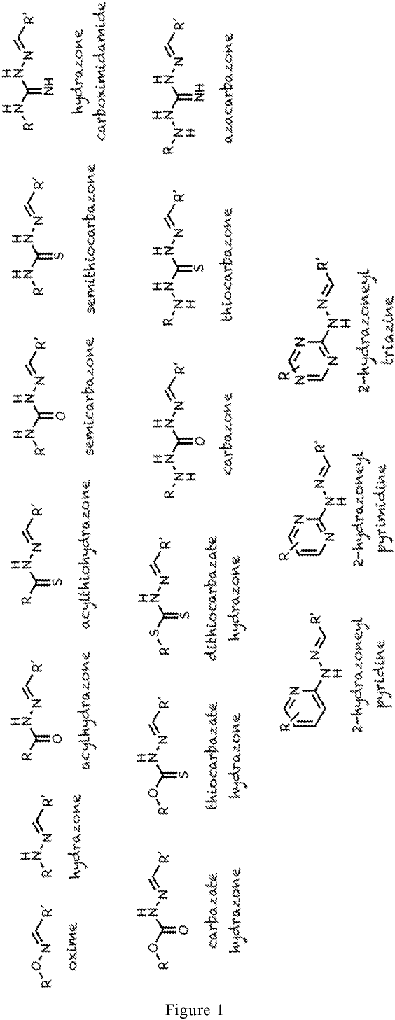

FIG. 11 shows the absorbance over time in the presence of the complex of E. coli strain MC4100 with either pAH-Bn or pAH-In. As shown in FIG. 1, the challenging conditions were handled surprisingly well by each complex.

The initial rates of increase in absorbance for the cultures including the complexes were higher than the bacteria alone.

Furthermore, the final yields of hydrolysis product indicated by the absorbance were significantly higher for the cultures including the complexes than for the bacteria alone.

Thus, it can be seen that cultures including complexes of a polymer according to formula (I) (i.e. including a complex of the polymer with the microorganism) can be used to perform chemical transformations.

Furthermore, cultures including complexes according to the invention can be less susceptible to challenging conditions than similar cultures without the polymer.

What is claimed is:

1-24. (canceled)

25. A microorganism-polymer complex comprising cells of a microorganism and a polymer comprising groups as defined by formula (Ia) and/or formula (Ib): 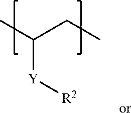 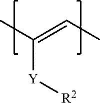
wherein:
Y comprises an imine; and
R² comprises a C1-30 group.

26. The microorganism-polymer complex of claim 25, wherein the complex is obtainable by a method comprising exposing, in an aqueous medium, cells of the microorganism to the polymer.

27. The microorganism-polymer complex of claim 25, wherein the imine is part of a group selected from the list consisting of hydrazone, oxime, acyl hydrazone, acyl thiohydrazone, semicarbazone, semithiocarbazone, hydrazone carboximidamide, carbazate hydrazone, thiocarbazate hydrazone, dithiocarbazate hydrazone, carbazone, thiocarbazone, azacarbazone, 2-hydrazoneyl pyridine, 2-hydrazoneyl pyrimidine and 2-hydrazoneyl triazine.

28. The microorganism-polymer complex of claim 27, wherein the imine is part of an acyl hydrazone.

29. The microorganism-polymer complex of claim 25, wherein Y further comprises one or more groups selected from the list consisting of: amide (such as a secondary or tertiary amide), ester, amine (such as a secondary or tertiary amine), ether, dialkyl peroxide, thioether, disulfide, sulfoxide, sulfone, sulfonamide, sulfonate ester, thioketone, thioester, phosphine, phosphonate ester, phosphate ester, boronic ester, borinic ester, borane, ketone, carbamate, carbonate, carboxylic acid anhydride, urea, ketal, acetal, orthoester, orthocarbonate, imide, diimide, hydrazine, hydroxylamine, 1,2,3-triazole, alkyl, alkenyl, alkynyl, aryl and heterocyclyl.

30. The microorganism-polymer complex of claim 29, wherein the list consists of amine, ether, aryl, heteroaryl and alkyl.

31. The microorganism-polymer complex of claim 25, wherein R² comprises a C3-30 group.

32. The microorganism-polymer complex of claim 31, wherein R² comprises a C5-30 group.

33. The microorganism-polymer complex of claim 32, wherein R² comprises a C5-30 aryl or heteroaryl group.

34. The microorganism-polymer complex of claim 25, wherein R² is selected from the list consisting of: 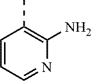 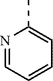 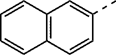 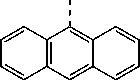 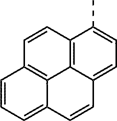 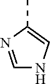 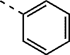 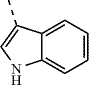 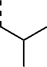
with the proviso that if the group of the polymer is of formula (Ia′): 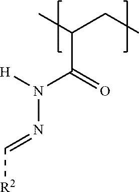
then R² can be selected from the list consisting of: 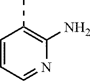 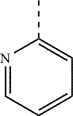 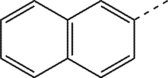 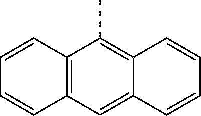 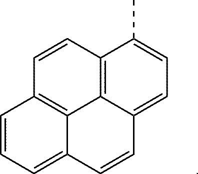
.

35. The microorganism-polymer complex of claim 25, wherein the polymer and/or the cells are in a dispersed state in the aqueous medium.

36. The microorganism-polymer complex of claim 25, wherein the clogD and/or clogP of the R² group is -2.0 or higher.

37. The microorganism-polymer complex of claim 25, wherein the microorganism is a bacteria or a fungi.

38. The microorganism-polymer complex of claim 37, wherein the microorganism is a bacteria.

39. A product comprising a microorganism-polymer complex according to claim 25, wherein the product is:
a) a composition for oral administration;
b) a composition for topical application; or
c) a coated article comprising an article coated with the microorganism-polymer complex.

40. The product of claim 39, wherein option c) applies and wherein the article is:
a) made of a plastic, a glass, a metal, or a natural material; or
b) is a live object.

41. The product of claim 40, wherein the article is a seed.

42. A method for producing a chemical and/or biological product, the method comprising exposing a substrate to a microorganism-polymer complex as defined by claim 25.

43. The method of claim 42, wherein the chemical and/or biological product is a medicament, an intermediate of a medicament, an organic molecule, a protein binding fragment, an antibody, a fine chemical and/or a bulk chemical.

44. A method of dispersing a microorganism-polymer complex, wherein the microorganism-polymer complex is as defined by claim 25, and wherein the method comprises the step of cleaving the polymer.

45. The method of claim 44, wherein the polymer is cleaved by hydrolysis.
